(19) United States
(12) Patent Application Publication
(10) Pub. No.: US 2007/0135357 A1
(43) Pub. Date: Jun. 14, 2007

(54) Anti-hypercholesterolemic compounds

(76) Inventors: Heather L. Sings, Rahway, NJ (US); Feroze Ujjainwalla, Scotch Plains, NJ (US); Malcolm MacCoss, Freehold, NJ (US); Robert W. Myers, Cresskill, NJ (US)

Correspondence Address: MERCK AND CO., INC, P O BOX 2000, RAHWAY, NJ, 07065-0907, US

(21) Appl. No.: 10/577,204

(22) PCT Filed: Oct. 27, 2004
(86) PCT No.: PCT/US04/35845

§ 371 Date: Apr. 26, 2006

Related U.S. Application Data
(60) Provisional application No. 60/515,842, filed on Oct. 30, 2003.

(51) Int. Cl. A61K 31/7052; A61K 31/397; C07D 409/02; C07H 17/02

(52) U.S. Cl. 514/25; 514/210.02; 540/200; 536/17.4

(57) ABSTRACT

The instant invention provides novel cholesterol absorption inhibitors of Formula I and the pharmaceutically acceptable salts and esters thereof. The compounds are useful for lowering plasma cholesterol levels, particularly LDL cholesterol, and for treating and preventing atherosclerosis and atherosclerotic disease events.

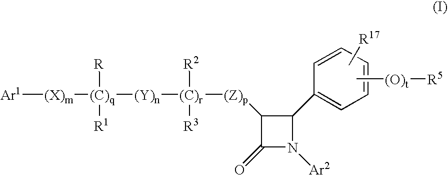

### BACKGROUND OF THE INVENTION

The instant invention relates to substituted 2-azetidinones and the pharmaceutically acceptable salts and esters thereof, and to their use alone or in combination with other active agents to treat hypercholesterolemia and for preventing, halting or slowing the progression of atherosclerosis and related conditions and disease events.

It has been clear for several decades that elevated blood cholesterol is a major risk factor for coronary heart disease, and many studies have shown that the risk of CHD events can be reduced by lipid-lowering therapy. Prior to 1987, the lipid-lowering armamentarium was limited essentially to a low saturated fat and cholesterol diet, the bile acid sequestrants (cholestyramine and colestipol), nicotinic acid (niacin), the fibrates and probucol. Unfortunately, all of these treatments have limited efficacy or tolerability, or both. Substantial reductions in LDL (low density lipoprotein) cholesterol accompanied by increases in HDL (high density lipoprotein) cholesterol could be achieved by the combination of a lipid-lowering diet and a bile acid sequestrant, with or without the addition of nicotinic acid. However, this therapy is not easy to administer or tolerate and was therefore often unsuccessful except in specialist lipid clinics. The fibrates produce a moderate reduction in LDL cholesterol accompanied by increased HDL cholesterol and a substantial reduction in triglycerides, and because they are well tolerated these drugs have been more widely used. Probucol produces only a small reduction in LDL cholesterol and also reduces HDL cholesterol, which, because of the strong inverse relationship between HDL cholesterol level and CHD risk, is generally considered undesirable. With the introduction of lovastatin, the first inhibitor of HMG-CoA reductase to become available for prescription in 1987, for the first time physicians were able to obtain large reductions in plasma cholesterol with very few adverse effects.

Recent studies have unequivocally demonstrated that lovastatin, simvastatin and pravastatin, all members of the HMG-CoA reductase inhibitor class, slow the progression of atherosclerotic lesions in the coronary and carotid arteries. Simvastatin and pravastatin have also been shown to reduce the risk of coronary heart disease events, and in the case of simvastatin a highly significant reduction in the risk of coronary death and total mortality has been shown by the Scandinavian Simvastatin Survival Study. This study also provided some evidence for a reduction in cerebrovascular events. Despite the substantial reduction in the risk of coronary morbidity and mortality achieved by simvastatin, the risk is still substantial in the treated patients. For example, in the Scandinavian Simvastatin Survival Study, the 42% reduction in the risk of coronary death still left 5% of the treated patients to die of their disease over the course of this 5 year study. Further reduction of risk is clearly needed.

A more recent class of anti-hyperlipidemic agents that has emerged includes inhibitors of cholesterol absorption. Ezetimibe, the first compound to receive regulatory approval in this class, is currently marketed in the U.S. under the tradename ZETIA®. Ezetimibe has the following chemical structure and is described in U.S. Pat. Nos. Re. 37721 and 5,846,966:

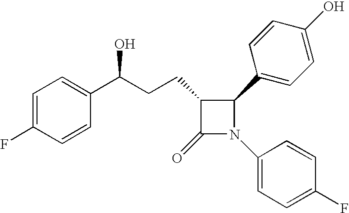

Additional cholesterol biosynthesis inhibitors are described in WO2002/066464 A1 (applied for by Kotobuki Pharmaceutical Co.), and US2002/0137689 A1 (Glombik et al.). WO2002/066464 A1 discloses hypolipidemic compounds of general formula 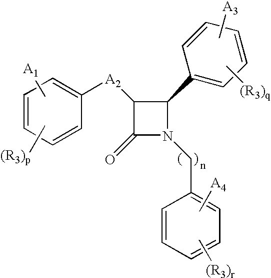
wherein, among other definitions, A1, A3 and A4 can be 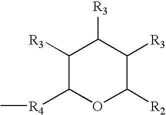
and wherein R2 is —CH₂OH, —CH₂OC(O)—R₁, or —CO₂R₁; R₃ is —OH or —OC(O)R₁, and R₄ is —(CH₂)ₖR₅(CH₂)ᵢ— where k and i are zero or integers of one or more, and k+i is an integer of 10 or less; and R₅ is a single bond, —CH═CH—, —OCH₂—, carbonyl or —CH(OH).

US2002/0137689 A1 discloses hypolipidemic compounds of general formula 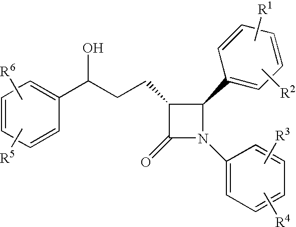
wherein, among other definitions, R¹, R², R³, R⁴, R⁵, R⁶ independently of one another can be (C₀-C₃₀)-alkylene-(LAG), where one or more carbon atoms of the alkylene radical may be replaced by —O—, —(C═O)—, —CH═CH—, —C≡C—, —N((C₁-C₆)-alkyl)-, —N((C₁-C₆)-alkylphenyl) or —NH—; and (LAG) is a sugar residue, disugar residue, trisugar residue, tetrasugar residue; a sugar acid, or an amino sugar.

In the ongoing effort to discover novel treatments for hyperlipidemia and atherosclerotic process, the instant invention provides novel cholesterol absorption inhibitors, described below.

### SUMMARY OF THE INVENTION

One object of the instant invention provides novel cholesterol absorption inhibitors of Formula I 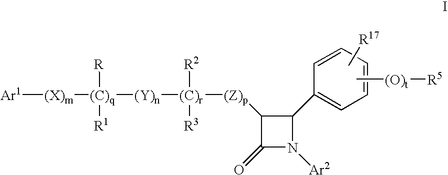
and the pharmaceutically acceptable salts and esters thereof.

A second object of the instant invention is to provide a method for inhibiting cholesterol absorption comprising administering a therapeutically effective amount of a compound of Formula I to a patient in need of such treatment.

Another object is to provide a method for reducing plasma cholesterol levels, especially LDL-cholesterol, and treating hypercholesterolemia comprising administering a therapeutically effective amount of a compound of Formula I to a patient in need of such treatment.

As a further object, methods are provided for preventing or reducing the risk of developing atherosclerosis, as well as for halting or slowing the progression of atherosclerotic disease once it has become clinically evident, comprising the administration of a prophylactically or therapeutically effective amount, as appropriate, of a compound of Formula I to a patient who is at risk of developing atherosclerosis or who already has atherosclerotic disease.

Another object of the present invention is the use of the compounds of the present invention for the manufacture of a medicament useful in treating, preventing or reducing the risk of developing these conditions.

Other objects of this invention are to provide processes for making the compounds of Formula I and to provide novel pharmaceutical compositions comprising these compounds. Additional objects will be evident from the following detailed description.

### DETAILED DESCRIPTION OF THE INVENTION

The novel cholesterol absorption inhibitors of the instant invention are compounds of Formula I 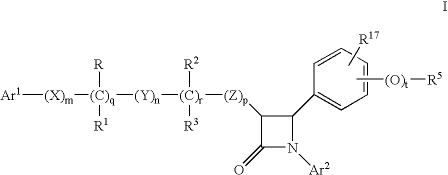
and the pharmaceutically acceptable salts and esters thereof, wherein
Ar¹ and Ar² are independently selected from the group consisting of aryl and R⁴-substituted aryl;
X, Y and Z are independently selected from the group consisting of —CH₂—, —CH(C₁₋₆alkyl)- and —C(C₁₋₆alkyl)₂—;
R is selected from the group consisting of —OR⁶, —O(CO)R⁶, —O(CO)OR⁹,
—O(CO)NR⁶R⁷, a sugar residue, a disugar residue, a trisugar residue and a tetrasugar residue;
R1 is selected from the group consisting of hydrogen, C₁₋₆alkyl and aryl or R and R¹ together are oxo;
R² is selected from the group consisting of —OR⁶, —O(CO)R⁶, —O(CO)OR⁹ and —O(CO)NR⁶R⁷;
R³ is selected from the group consisting of hydrogen, —C₁₋₆alkyl and aryl or R² and R³ together are oxo;
q, r and t are each independently selected from 0 and 1; m, n and p are each independently selected from 0, 1, 2, 3 and 4; provided that at least one of q and r is 1, and the sum of m, n, p, q are r is 1, 2, 3, 4, 5 or 6; and provided that when p is 0 and r is 1, the sum of m, q and n is 1, 2, 3, 4, or 5;
R⁴ is 1-5 substituents independently selected at each occurrence from the group consisting of: —OR⁶, —O(CO)R⁶, —O(CO)OR⁹, —O—C₁₋₅alkyl-OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O—C₁₋₁₀alkyl-COOR⁶, —O—C₁₋₁₀alkyl-CONR⁶R⁷ and fluoro;
R⁶, R⁷ and R⁸ are independently selected at each occurrence from the group consisting of hydrogen, C₁₋₆alkyl, aryl and aryl-substituted C₁₋₁₆alkyl;
R⁹ is independently selected from the group consisting of C₁₋₆alkyl, aryl and aryl-substituted C₁₋₆alkyl;
R⁵ is selected from
   (a) —R¹⁰—R¹¹, wherein R¹⁰ is selected from the group consisting of —S—, —S(O)—, —SO₂— and —C₁₋₆ n-alkyl-substituted with one to three substituents selected from the group consisting of —C₁₋₆alkyl, —O(C₁₋₁₆alkyl), —CF₃, —OCF₃, —NR⁶R⁷ and —F;
   (b) —R¹²—R¹³, wherein R¹² is selected from (i) a bond and (ii) a member selected from the group consisting of —S—, —S(O)—, —SO₂—, —C₁₋₆ n-alkyl-, and —C₁₋₆ n-alkyl-N(R⁶)—, wherein the alkyl group is unsubstituted or substituted with one to three substituents selected from the group consisting of —OH, oxo, —C₁₋₆alkyl, —O(C₁₋₆alkyl), —CF₃, —OCF₃, —NR⁶R⁷ and —F, and provided that when R¹² is a bond then t is 1;
R¹¹ is selected from the group consisting of a sugar residue, disugar residue, trisugar residue and tetrasugar residue;
R¹³ is selected from the group consisting of:
   (a) a thiosugar residue selected from the group consisting of: 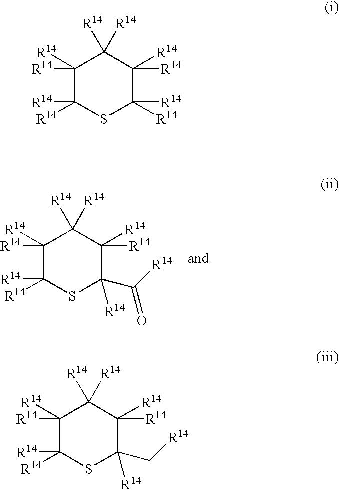
   wherein R¹⁴ is independently selected at each occurrence from (i) a linking bond and (ii) a member of the group consisting of —F, —H, —C₁₋₆alkyl, —OC₁₋₆alkyl, —OCF₃, —OH, —O—PG, —OR¹¹ and —OR¹³, and provided that: (A) one and only one occurrence of R¹⁴ is a linking bond, (B) an R¹⁴ adjacent to a carbonyl is not —F, and (C) no more than one occurrence of R¹⁴ is selected from —OR¹¹ and —OR¹³;
   (b) a fluorosugar residue selected from the group consisting of: 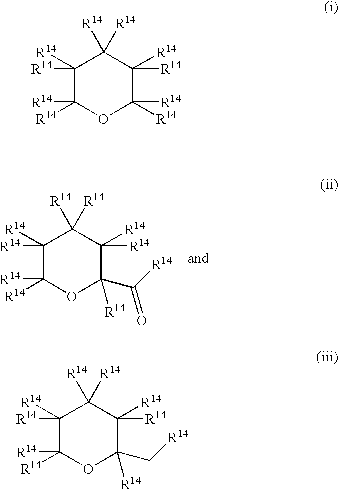
   wherein R¹⁴ is independently selected at each occurrence from (i) a linking bond and (ii) a member of the group consisting of —F, —H, —C₁₋₆alkyl, —OC₁₋₆alkyl, —OCF₃, —OH, —O—PG, —OR¹¹ and —OR¹³, and provided that: (A) one and only one occurrence of R¹⁴ is a linking bond, (B) at least one occurrence of R¹⁴ is —F, (C) an R¹⁴ adjacent to a carbonyl is not —F, and (D) no more than one occurrence of R¹⁴ is selected from —OR¹¹ and —OR¹³; 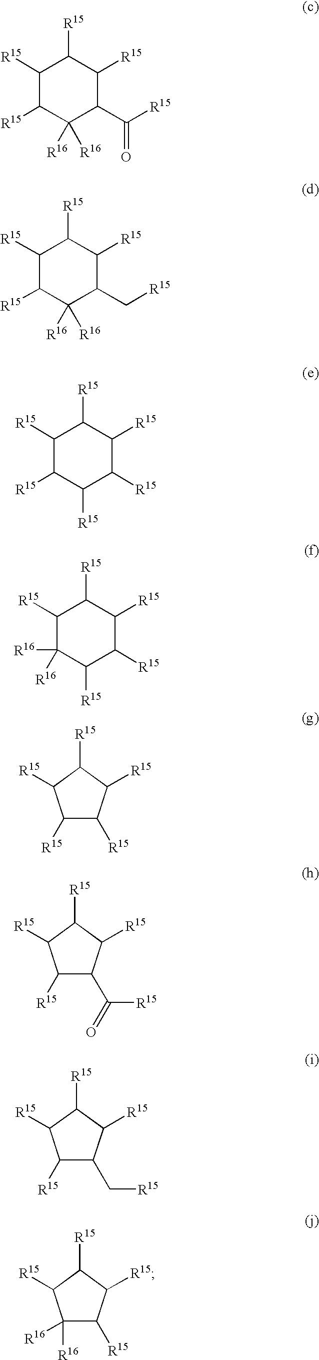 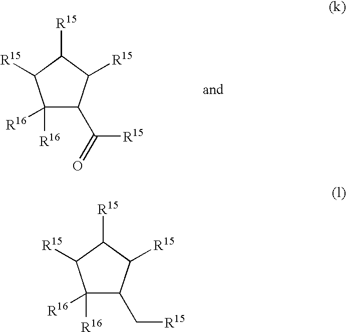
   wherein R¹⁵ is independently selected at each occurrence from (i) a linking bond and (ii) a member of the group consisting of —H, —C₁₋₆alkyl, —OC₁₋₆alkyl, —OCF₃, —OH, —O—PG, —OR¹¹, —OR¹³, —SR¹¹, —SR¹³, —NR⁶R¹¹ and —NR⁶R¹³, and provided that: (A) one and only one occurrence of R¹⁵ is a linking bond and (B) no more than one occurrence of R¹⁵ is selected from —OR¹¹, —OR¹³, —SR¹¹, —SR¹³, —NR⁶R¹¹ and —NR⁶R¹³;
R¹⁶ is independently selected at each occurrence from the group consisting of —H and —F;
PG is a hydroxyl protecting group;
and provided that R⁵ is comprised of no more than four of any combination of sugar residues and members within the definition of R¹³ linked together. and
R¹⁷ is selected from the group consisting of —H, —OH, —C₁₋₆alkyl, —OC₁₋₆alkyl, —CF₃, —CN, —NR⁶R⁷ and halogen.

In one embodiment of Formula I, the —(O)t—R⁵ moiety is attached to the phenyl ring para to the azetidinone, and the R⁵ group is comprised of either —R¹⁰ or —R¹² and one or two of a combination of sugar residues and members within the definition of R¹³ linked together.

In a second embodiment of this invention are compounds of Formula Ia: 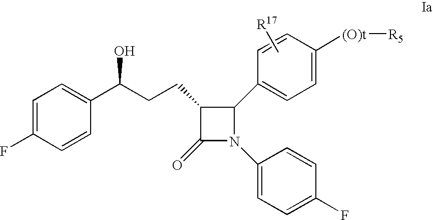
Within Formula Ia of the second embodiment, preferably the —(O)t—R⁵ moiety is attached to the phenyl ring para to the azetidinone, and the R⁵ group is comprised of one or two of a combination of sugar residues and members within the definition of R¹³ linked together.

In one class of both the first and second embodiments, t is one, R⁵ is —R¹²—R¹³, and R¹² is a bond; thus the —(O)t—R⁵ moiety in Formula I is equivalent to —OR¹³. In a first subclass of this class, R¹³ is a thiosugar. An example within the first subclass includes but is not limited to: 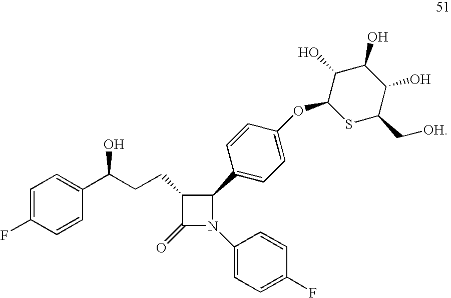
In a second subclass of this class, R¹³ is 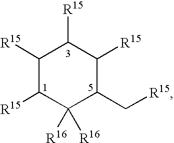
R¹⁵ at position 1 is a linking bond and all the remaining R¹⁵ groups are —OH; or R¹⁵ at position 1 is a linking bond, R¹⁵ at position 4 is —OR¹¹ and the remaining R¹⁵ groups are —OH. Examples of compounds within this subclass include but are not limited to:

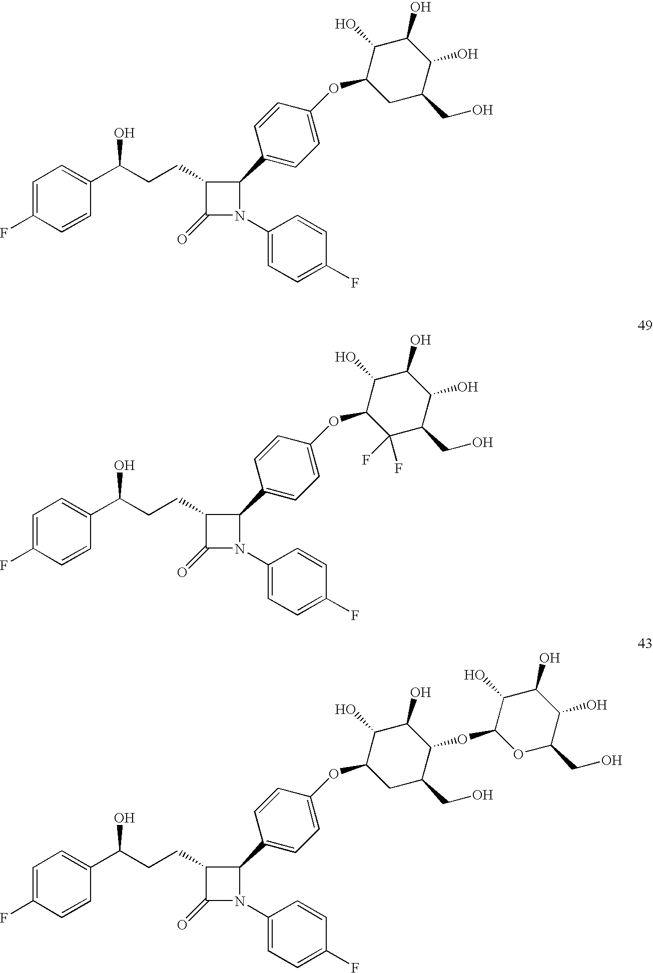

In a second class of both the first and second embodiments, t is zero and R⁵ is —R¹⁰—R¹¹; thus the —(O)t—R⁵ moiety in Formula I is equivalent to —R¹⁰—R¹¹. In a subclass of this class, R¹¹ is a sugar residue or a disugar residue. Preferred within this subclass are compounds wherein R¹⁰ is —S— or —CF₂—. Examples of compounds within this subclass include but are not limited to:

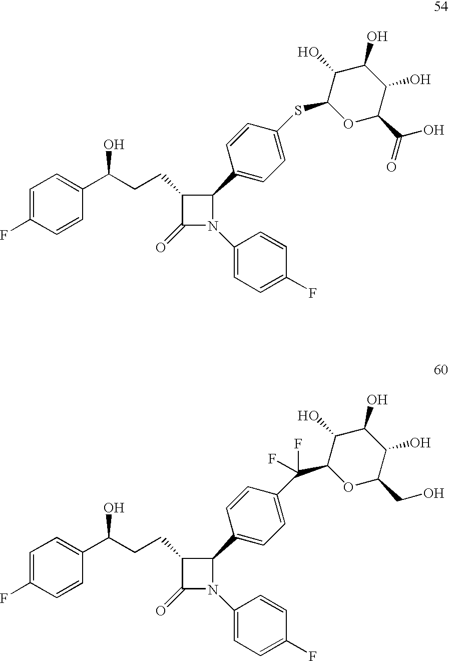

As used herein, the term “sugar residue” is intended to encompass monosaccharides which are derived from aldoses and ketoses which have 3-7 carbon atoms, which may be acyclic or cyclic and may belong to the D or L-series, and includes within its scope residues of amino sugars, sugar alcohols and sugar acids. As used herein, the term “sugar residue” does not include thiosugars or fluorosugars, which are defined separately herein. Amino sugars are monosaccharides in which an alcoholic hydroxy group has been replaced by an amino group.

The terms sugar, saccharide and carbohydrate may be used interchangeably. Most monosaccharides exist as cyclic hemiacetals or hemiketals, and may be in the α or β anomeric form Cyclic forms with a three-membered ring are called oxiroses, those with a four-membered ring oxetoses, those with a five-membered ring furanoses, with a six-membered ring pyranoses, with a seven-membered ring septanoses. Cyclic sugar residues are preferred, particularly 5-membered (furanose) and 6-membered (pyranose) rings.

Oligosaccharides are compounds in which monosaccharide units are joined by glycosidic linkages, including both oxygen and carbon glycosidic linkages. According to the number of units, they are called disaccharides, trisaccharides, tetrasaccharides, etc. Herein, disaccharide is also referred to as disugar, trisaccharide as trisugar and tetrasaccharide as tetrasugar.

As used herein, the term “thiosugar(s)” are cyclized monosaccharides in which the ring oxygen atom of the cyclic form of an aldose or ketose is replaced by sulfur. General examples of thiosugars encompassed within the scope of Formula I include: 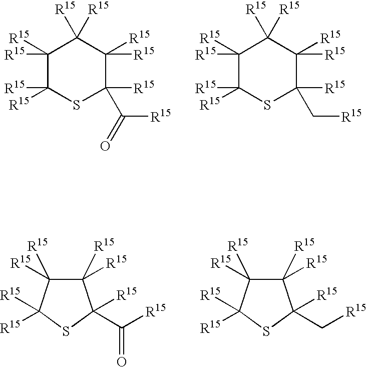
wherein R¹⁵ is defined above. Examples of thiosugars include, but are not limited to: 5-thio-glucopyranose, 5-thio-mannopyranose, 5-thio-galactopyranose, and 5-thio-fucopyranose. Cyclic 5-membered (furanose) and 6-membered (pyranose) thiosugars are preferred.

The fluorosugar residues encompassed within Formula I are 6-membered cyclic sugars substituted on the ring with one or more of fluoro.

Suitable protecting groups (designated as “PG” in the definitions above) for the hydroxyl groups of R¹¹ (i.e., sugars, disugars, trisugars, and tetrasugars) and R¹³ (i.e., thiosugars, fluorosugars and additional cycloalkyl rings defined therein) include but are not limited to those that are known to be useful as carbohydrate protecting groups, such as, for example benzyl, acetyl, benzoyl, tert-butyldiphenylsilyl, trimethylsilyl, para-methoxybenzyl, benzylidine, and methoxy methyl. Conditions required to selectively add and remove such protecting groups are found in standard textbooks such as Greene, T, and Wuts, P. G. M., Protective Groups in Organic Synthesis, John Wiley & Sons, Inc., New York, N.Y., 1999.

Within the definition of R¹³, one of R¹⁴ or R¹⁵, as appropriate, is a “linking bond.” As R¹³ is defined in Formula I, one or more residues selected from thiosugar residues, fluorosugar residues, cycloalkyl ring residues as defined in R¹³ (c) to (l) and sugar residues (i.e., monosaccharides) in any combination can be linked together one to the next, up to a maximum of four residues in the chain. For brevity, the cycloalkyl ring residues as defined in R¹³ (c) to (l) may be collectively referred to herein as “sugar mimetics.”

R⁵ may be comprised of R¹² connected to a single R¹³ residue selected from a thiosugar residue, a fluorosugar residue and a sugar mimetic (i.e., when none of the R¹⁴ groups are —OR¹¹ or —OR¹³, or none of the R¹⁵ groups are —OR¹³, —SR¹¹, —SR¹³, —NR⁶R¹¹ or —NR⁶R¹³). When R⁵ is comprised of R¹² connected to a single R¹³ unit, the linking bond of R¹³ connects to R¹²; or when R¹² is a bond, then “t” must be one and the linking bond of R¹³ connects to —(O)— as exemplified below: 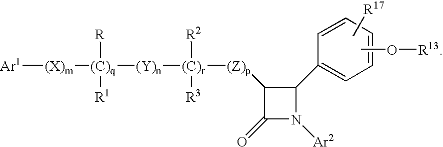
When a second residue, selected from a sugar residue, a thiosugar residue, a fluorosugar residue and a sugar mimetic, is connected to the first R¹³ unit (i.e., when one R¹⁴ is —OR¹¹ or —OR¹³, or one R¹⁵ is —OR¹³, —SR¹¹, —SR¹³, —NR⁶R¹¹ or —NR⁶R¹³ in the first R¹³ residue), then the linking bond of the second residue connects the second residue to the first R¹³ unit, and so on for additional residues up to four in the chain. If four residues are in the chain, then the fourth reside cannot be substituted with any of —OR¹¹, —OR¹³, —OR¹³, —SR¹¹, —SR¹³, —NR⁶R¹¹ and —NR⁶R¹³.

In choosing compounds of the present invention, one of ordinary skill in the art will recognize that the various substituents, i.e. R¹, R², etc., are to be chosen in conformity with well-known principles of chemical structure connectivity and stability. When any variable (e.g., R¹, R², etc.) occurs more than one time in any constituent or in Formula I, its definition on each occurrence is independent of its definition at every other occurrence. Also, combinations of substituents and/or variables are permissible only if such combinations result in stable compounds.

Compounds of Formula I may contain one or more asymmetric centers and can thus occur as racemates and racemic mixtures, single enantiomers, enantiomeric mixtures, diastereomeric mixtures and individual diastereomers. The present invention is meant to comprehend all such isomeric forms of the compounds of Formula I. All such isomeric forms of the compounds of Formula I are included within the scope of this invention. Some of the compounds described herein contain olefinic double bonds, and unless specified otherwise, are meant to include both E and Z geometric isomers. Furthermore, some of the crystalline forms for compounds of the present invention may exist as polymorphs and as such are intended to be included in the present invention. In addition, some of the compounds of the instant invention may form solvates with water or common organic solvents. Such solvates are also encompassed within the scope of this invention.

Herein, the term “pharmaceutically acceptable salts” shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenzimidazole, diethylamine, piperazine, morpholine, 2,4,4-trimethyl-2-pentamine and tris(hydroxymethyl)aminomethane.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, furnaric, gluconic, glutamic, hydrobromic, hydrochloric, isethionic, lactic, maleic, malic, mandelic, methamesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

Examples of pharmaceutically acceptable esters include, but are not limited to, —C1-4alkyl and —C1-4alkyl substituted with phenyl-, dimethylamino-, and acetylamino. “C1-4alkyl” herein includes straight or branched aliphatic chains containing from 1 to 4 carbon atoms, for example methyl, ethyl, n-propyl, n-butyl, iso-propyl, sec-butyl and tert-butyl.

The term “patient” includes mammals, especially humans, who use the instant active agents for the prevention or treatment of a medical condition. Administering of the drug to the patient includes both self-administration and administration to the patient by another person. The patient may be in need of treatment for an existing disease or medical condition, or may desire prophylactic treatment to prevent or reduce the risk for diseases and medical conditions affected by inhibition of cholesterol absorption.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. Particularly, the dosage a patient receives can be selected so as to achieve the amount of LDL cholesterol lowering desired; the dosage a patient receives may also be titrated over time in order to reach a target LDL level. The dosage regimen utilizing a compound of the instant invention is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition.

The compounds of the instant invention are cholesterol absorption inhibitors and are useful for reducing plasma cholesterol levels, particularly reducing plasma LDL cholesterol levels, when used either alone or in combination with another active agent, such as an anti-atherosclerotic agent, and more particularly a cholesterol biosynthesis inhibitor, for example an HMG-CoA reductase inhibitor. Thus the instant invention provides methods for inhibiting cholesterol absorption and for treating lipid disorders including hypercholesterolemia, comprising administering a therapeutically effective amount of a compound of Formula I to a person in need of such treatment. Further provided are methods for preventing or reducing the risk of developing atherosclerosis, as well as for halting or slowing the progression of atherosclerotic disease once it has become clinically evident, comprising the administration of a prophylactically or therapeutically effective amount, as appropriate, of a compound of Formula I to a mammal who is at risk of developing atherosclerosis or who already has atherosclerotic disease.

Atherosclerosis encompasses vascular diseases and conditions that are recognized and understood by physicians practicing in the relevant fields of medicine. Atherosclerotic cardiovascular disease including restenosis following revascularization procedures, coronary heart disease (also known as coronary artery disease or ischemic heart disease), cerebrovascular disease including multi-infarct dementia, and peripheral vessel disease including erectile dysfunction are all clinical manifestations of atherosclerosis and are therefore encompassed by the terms “atherosclerosis” and “atherosclerotic disease.”

A compound of Formula I may be administered to prevent or reduce the risk of occurrence, or recurrence where the potential exists, of a coronary heart disease event, a cerebrovascular event, and/or intermittent claudication. Coronary heart disease events are intended to include CHD death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events, and intermittent claudication. It is intended that persons who have previously experienced one or more non-fatal atherosclerotic disease events are those for whom the potential for recurrence of such an event exists.

Accordingly, the instant invention also provides a method for preventing or reducing the risk of a first or subsequent occurrence of an atherosclerotic disease event comprising the administration of a prophylactically effective amount of a compound of Formula I to a patient at risk for such an event. The patient may or may not have atherosclerotic disease at the time of administration, or may be at risk for developing it.

Persons to be treated with the instant therapy include those at risk of developing atherosclerotic disease and of having an atherosclerotic disease event. Standard atherosclerotic disease risk factors are known to the average physician practicing in the relevant fields of medicine. Such known risk factors include but are not limited to hypertension, smoking, diabetes, low levels of high density lipoprotein (HDL) cholesterol, and a family history of atherosclerotic cardiovascular disease. Published guidelines for determining those who are at risk of developing atherosclerotic disease can be found in: National Cholesterol Education Program, Second report of the Expert Panel on Detection, Evaluation, and Treatment of High Blood Cholesterol in Adults (Adult Treatment Panel II), National Institute of Health, National Heart Lung and Blood Institute, NIH Publication No. 93-3095, September 1993; abbreviated version: Expert Panel on Detection, Evaluation, and Treatment of High Blood Cholesterol in Adults, Summary of the second report of the national cholesterol education program (NCEP) Expert Panel on Detection, Evaluation, and Treatment of High Blood Cholesterol in Adults (Adult Treatment Panel II), JAMA, 1993, 269, pp. 3015-23. People who are identified as having one or more of the above-noted risk factors are intended to be included in the group of people considered at risk for developing atherosclerotic disease. People identified as having one or more of the above-noted risk factors, as well as people who already have atherosclerosis, are intended to be included within the group of people considered to be at risk for having an atherosclerotic disease event.

The oral dosage amount of the compound of Formula I, is from about 0.1 to about 30 mg/kg of body weight per day, preferably about 0.1 to about 15 mg/kg of body weight per day. For an average body weight of 70 kg, the dosage level is therefore from about 5 mg to about 1000 mg of drug per day. However, dosage amounts will vary depending on factors as noted above, including the potency of the particular compound. Although the active drug of the present invention may be administered in divided doses, for example from two to four times daily, a single daily dose of the active drug is preferred. As examples, the daily dosage amount may be selected from, but not limited to, 5 mg, 10 mg, 15 mg, 20 mg, 25 mg, 40 mg, 50 mg, 75 mg, 80 mg, 100 mg and 200 mg.

The active drug employed in the instant therapy can be administered in such oral forms as tablets, capsules, pills, powders, granules, elixirs, tinctures, suspensions, syrups, and emulsions. Oral formulations are preferred.

For compounds of Formula I, administration of the active drug can be via any pharmaceutically acceptable route and in any pharmaceutically acceptable dosage form. This includes the use of oral conventional rapid-release, time controlled-release and delayed-release (such enteric coated) pharmaceutical dosage forms. Additional suitable pharmaceutical compositions for use with the present invention are known to those of ordinary skill in the pharmaceutical arts; for example, see Remington's Pharmaceutical Sciences, Mack Publishing Co., Easton, Pa.

In the methods of the present invention, the active drug is typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with a non-toxic, pharmaceutically acceptable, inert carrier such as lactose, starch, sucrose, glucose, modified sugars, modified starches, methyl cellulose and its derivatives, dicalcium phosphate, calcium sulfate, mannitol, sorbitol and other reducing and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents and coloring and flavoring agents can also be incorporated into the mixture. Stabilizing agents such as antioxidants, for example butylated hydroxyanisole (BHA), 2,6-di-tert-butyl-4-methylphenol (BHT), propyl gallate, sodium ascorbate, citric acid, calcium metabisulphite, hydroquinone, and 7-hydroxycoumarin, particularly BHA, propyl gallate and combinations thereof, can also be added to stabilize the dosage forms. When a compound of Formula I is formulated together with an HMG-CoA reductase inhibitor such as simvastatin, the use of at least one stabilizing agent is preferred in the composition. Other suitable components include gelatin, sweeteners, natural and synthetic gums such as acacia, tragacanth or alginates, carboxymethylcellulose, polyethylene glycol, waxes and the like.

The active drug can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Active drug may also be delivered by the use of monoclonal antibodies as individual carriers to which the compound molecules are coupled. Active drug may also be coupled with soluble polymers as targetable drug carriers. Such polymers can include polyvinyl-pyrrolidone, pyran copolymer, polyhydroxy-propyl-methacrylamide-phenol, polyhydroxy-ethyl-aspartamide-phenol, or polyethyleneoxide-polylysine substituted with palmitoyl residues. Furthermore, active drug may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

The instant invention also encompasses a process for preparing a pharmaceutical composition comprising combining a compound of Formula I with a pharmaceutically acceptable carrier. Also encompassed is the pharmaceutical composition which is made by combining a compound of Formula I with a pharmaceutically acceptable carrier.

In a broad embodiment, any suitable additional active agent or agents may be used in combination with the compound of Formula I in a single dosage formulation, or may be administered to the patient in a separate dosage formulation, which allows for concurrent or sequential administration of the active agents. One or more additional active agents may be administered with a compound of Formula I. The additional active agent or agents can be lipid modifying agents, particularly a cholesterol biosynthesis inhibitor, or agents having other pharmaceutical activities, or agents that have both lipid-modifying effects and other pharmaceutical activities. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), dihydroxy open-acid simvastatin, particularly the ammonium or calcium salts thereof, pravastatin, particularly the sodium salt thereof (see U.S. Pat. No. 4,346,227), fluvastatin, particularly the sodium salt thereof (see U.S. Pat. No. 5,354,772), atorvastatin, particularly the calcium salt thereof (see U.S. Pat. No. 5,273,995), pitavastatin also referred to as NK-104 (see PCT international publication number WO 97/23200) and rosuvastatin, (CRESTOR®; see U.S. Pat. No. 5,260,440, and Drugs of the Future, 1999, 24 (5), pp. 511-513); HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; probucol; niacin; cholesterol absorption inhibitors such as SCH-58235, which is described in U.S. Pat. Nos. 5,767,115 and 5,846,966; bile acid sequestrants; LDL (low density lipoprotein) receptor inducers; platelet aggregation inhibitors, for example glycoprotein IIb/IIIa fibrinogen receptor antagonists and aspirin; human peroxisome proliferator activated receptor gamma (PPARγ) agonists including the compounds commonly referred to as glitazones for example troglitazone, pioglitazone and xosiglitazone and, including those compounds included within the structural class known as thiazolidinediones as well as those PPARγ agonists outside the thiazolidinedione structural class; PPARα agonists such as clofibrate, fenofibrate including micronized fenofibrate, and gemfibrozil; PPAR dual α/γ agonists; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; calcium channel blockers such as nifedipine and diltiazam; endothelian antagonists; agents that enhance ABC1 gene expression; FXR ligands including both inhibitors and agonists; and LXR ligands including both inhibitors and agonists of all subtypes of this receptor, e.g., LXRα and LXRβ; bisphosphonate compounds such as a lendronate sodium; and cyclooxygenase-2 inhibitors such as rofecoxib and celecoxib. Additionally, the compound of Formula Is of this invention, for example compound I, may be used in combination with anti-retroviral therapy in AIDS infected patients to treat lipid abnormalities associated with such treatment, for example but not limited to their use in combination with HIV protease inhibitors such as indinavir, nelfinavir, ritonavir and saquinavir.

A therapeutically or prophylactically effective amount, as appropriate, of a compound of Formula I can be used for the preparation of a medicament useful for inhibiting cholesterol absorption, as well as for treating and/or reducing the risk for diseases and conditions affected by inhibition of cholesterol absorption, such as treating lipid disorders, preventing or reducing the risk of developing atherosclerotic disease, halting or slowing the progression of atherosclerotic disease once it has become clinically manifest, and preventing or reducing the risk of a first or subsequent occurrence of an atherosclerotic disease event. For example, the medicament may be comprised of about 5 mg to about 1000 mg of a compound of Formula I. The medicament comprised of a compound of Formula I may also be prepared with one or more additional active agents, such as those described supra.

The compounds of structural Formula I of the present invention can be prepared according to the procedures of the following Schemes and Examples, using appropriate materials, and are further exemplified by specific examples which follow. Moreover, by utilizing the procedures described herein, one of ordinary skill in the art can readily prepare additional compounds of the present invention claimed herein. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted.

The term “appropriate volume” means the amount of solvent added to a reaction mixture such that the reaction is performed at a synthetically useful concentration. The synthetically useful concentration is dependant on the nature of the reaction, and should be clear to those who are skilled in the art of organic synthesis. The term “variety of chromatographic techniques” refers to those techniques which are typically utilized by those engaged in synthetic organic chemistry. These techniques include, but are not limited to: High Performance Liquid Chromatography (including normal- reversed- and chiral-phase); Super Critical Fluid Chromatography; preparative Thin Layer Chromatography; flash chromatography with silica gel or reversed-phase silica gel; ion-exchange chromatography; and radial chromatography. The term “until the reaction is deemed complete” refers to the point at which the operator determines that the reaction should be terminated. The operator may base this time point on the disappearance of starting material or on the formation of product, or acceptable conversion of starting material to product, using any number of the methods know to one who is skilled in the art. These methods include, but are not limited to: Tlc or HPLC coupled to mass spectrometry (LC/MS).

Some abbreviations used herein include:
Ac Acyl (CH₃C(O)—)
9-BBN 9-Borabicyclo[3.3.1]nonane
Bn benzyl
calc. Calculated
Celite Celite™ diatomaceous earth
DCM dichloromethane
DIPEA Diisopropylethylamine
DMAP 4-dimethylaminopyridine
equiv. Equivalent(s)
Et Ethyl
EtOAc Ethyl acetate
H Hour(s)
HPLC High performance liquid chromatography
Lg Leaving group
MOM methoxymethyl
Me Methyl
Min Minute(s)
m.p. Melting point
MS Mass spectrum
PMB Para-methoxybenzyl
Ph Phenyl
Pr Propyl
ⁱPr Isopropyl
p-TSA Para-toluenesulfonic acid
r.t. Room temperature
t Tert
TBAF Tetrabutylammonium fluoride
TBDMS Tert-butyldimethylsilyl
TBDPS Tert-butyldiphenylsilyl
Tf Triflate or trifuoromethanesulfonate
TFA Trifluoroacetic acid
THF Tetrahydrofuran
Tlc Thin layer chromatography

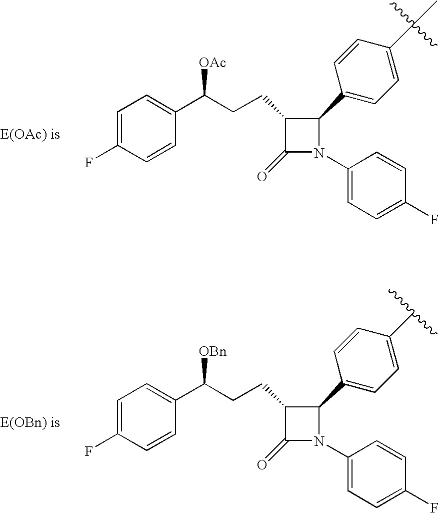

Reaction Schemes A-C illustrate the general methods employed in the synthesis of the compounds of the present invention of structural Formula I. All substituents are as defined above unless indicated otherwise.

Reaction Scheme A illustrates a preferred method for glycosylation to generate compounds of the general Formula I (4). Here, a thiol or a phenol of type 1, possessing the 2-azetidinone cholesterol absorption inhibitor backbone, serves as the glycosyl acceptor. The glycosyl donor is a compound of type 2, which may be derived from a suitably protected mono- or polysaccharide. Widely used glycosyl donors in oligosaccharide synthesis include trichloroacetimidates, thioethers, and halides. In the trichloroacetimidate method, the glycosylation reaction is generally promoted by catalytic use of an “activator” such as boron trifluoride diethyl etherate or trimethylsilyl trifluoromethanesulfonate. If the Lewis acid has the potential of reacting with the substrates or their protecting groups, milder metal salts such as cobalt(II) bromide, copper(II) trifluoromethanesulfonate, or silver trifluoromethanesulfonate can be used as alteratives (Whitfield, D. M.; Douglas, S. P. Glycoconjugate Journal, 1996, 13, 5) For thioether-based glycosyl donors, mercury salts or other thiophilic metal salts may be employed as activators. Methods for the formation of glycosidic bonds through the use of glycosyl donors and definitions relating to the concept of glycosyl donors/acceptors are well documented, and such methods can be found in Schmidt, R. R. Angew. Chem. Int. Ed. 1986, 25, 212; and Toshima, K.; Tatsuta, K. Chem. Rev. 1993, 93, 1503. Thus the glycosyl acceptor 1 is allowed to react with glycosyl donor 2, to generate a compound of type 3. The potential resulting mixture of α- and β-anomers can be separated by the chromatographic techniques discussed below. Furthermore, the potential anomeric mixture may be separated at this stage, or at later stages in the synthetic sequence, as deemed appropriate by the operator.

The next stage in the synthesis of compounds of type 4 involves deprotection of the carbohydrate unit. The use of protecting groups for the carbohydrate and carbohydrate derivatives such as those described herein, to facilitate the desired reaction and minimize undesired side-reactions, is well documented. Conditions required to add and remove protecting groups are found in standard textbooks such as Greene, T, and Wuts, P. G. M., Protective Gross in Organic Synthesis, John Wiley & Sons, Inc., New York, N.Y., 1999. Acetate, benzyl, p-methoxybenzyl, benzilidine, and tert-butyldiphenylsilyl are commonly used hydroxyl protecting groups in carbohydrate synthesis, and conditions for the selective removal of these groups are known to those skilled in the art. For example, if the hydroxyl groups of the mono- or polysaccharide unit in 3 are protected as the acetate ester, basic hydrolysis employing lithium or sodium hydroxide may effectively cleave this protecting group to generate compounds of type 4.

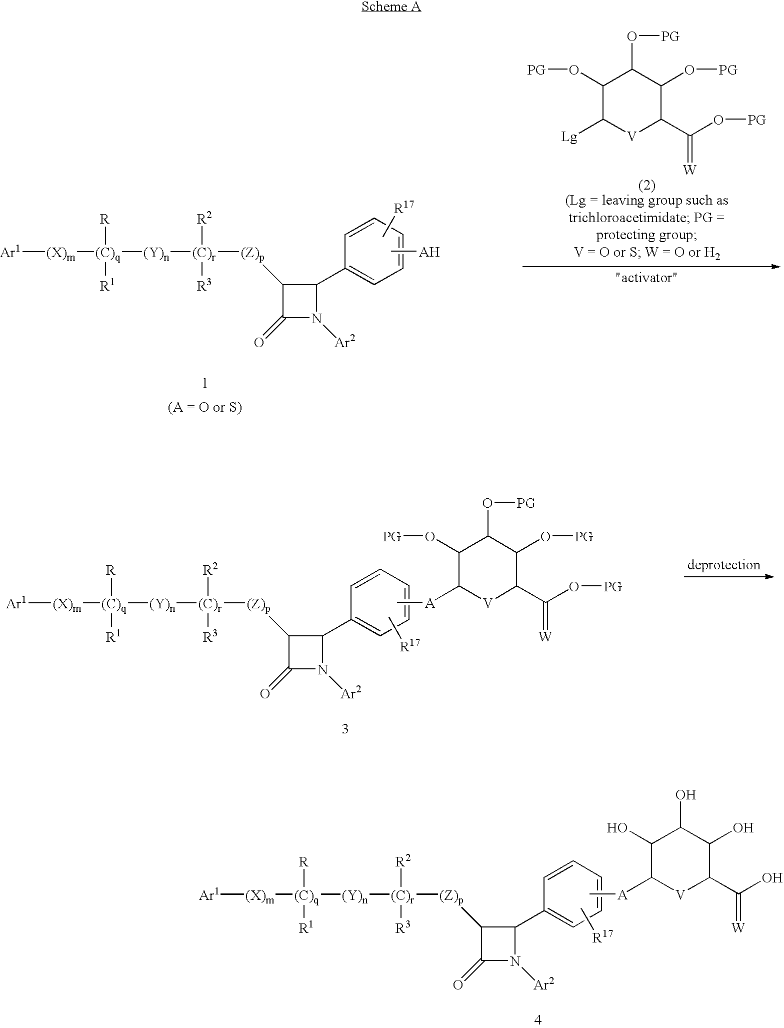

Reaction Scheme B illustrates a general strategy which may be employed to synthesize di-, tri- or tetrasaccharides of the general Formula I (8 and 9). One may envision two ways to generate such derivatives. In one method, a preconstructed poly-saccharide is attached to 1 using the standard glycosidic-bond forming methodologies as described above in Scheme A. However, in some instances, it may be desired to add additional carbohydrate units after the first sugar derivative has been attached to the 2-azetidinone core. This may be accomplished by first constructing a compound such as 5, using the methodology outlined in Scheme A, that incorporates an orthogonally protected sugar unit (Scheme B). The hydroxyl groups of the differentially protected sugar unit in 5 can then be selectively removed. For example, if the C4 hydroxyl of the sugar unit in 5 is protected as the para-methoxybenzyl (PMB) derivative, this protecting group can be selectively removed in the presence of the silyl and benzyl protecting groups with a reagent such as 2,3-dichloro-5,6-dicyano-1,4-benzoquinone (DDQ), to afford a compound of type 7. On the other hand, if one desires to attach additional sugar units to the C-6 hydroxyl of 5, the tert-butyldiphenylsilyl (TBDPS) protecting group may be selectively removed by reaction with a reagent such as TBAF to afford a compound of type 6. Coupling of a second sugar unit to either the C-4 or C-6 hydroxyl can then be accomplished as described in Scheme A to afford 8 or 9.

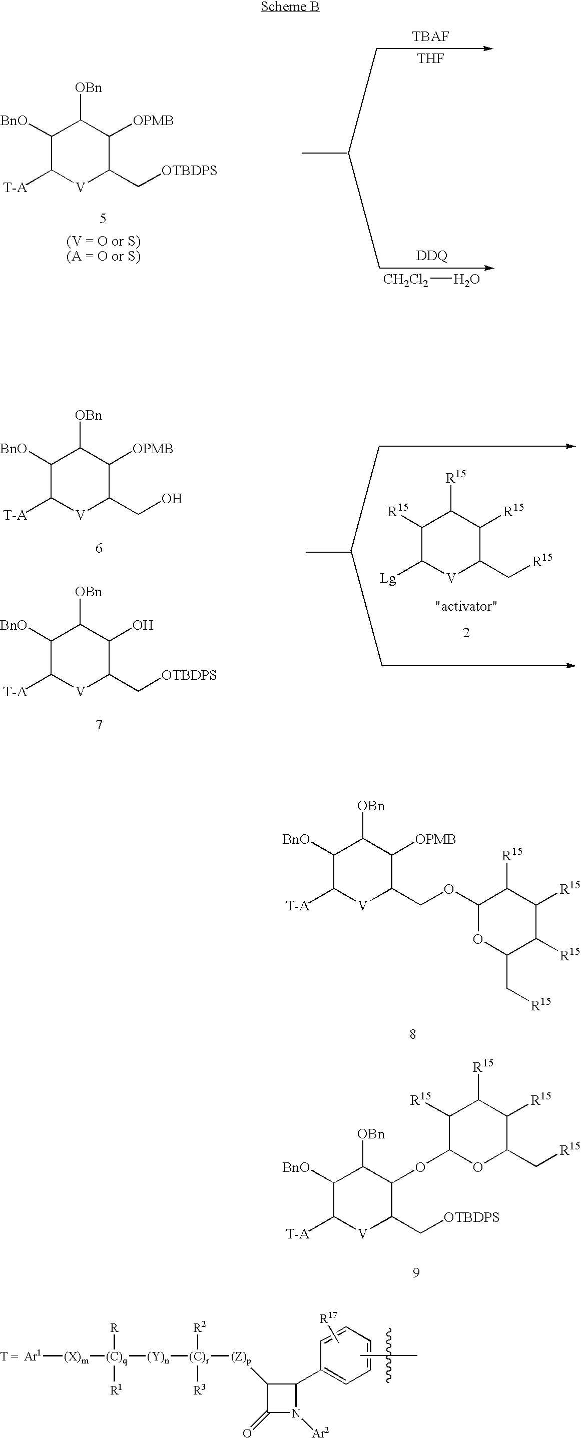

Reaction Scheme C illustrates a preferred method to synthesize compounds of the general Formula I (4), possessing an aryl C-glycosidic linkage. The sugar lactone precursors of type 10 can be purchased commercially or synthesized using the standard methodology know to those skilled in the art of carbohydrate chemistry. Examples of the synthesis of hydroxyl-protected sugar lactones can be found in the following references: Shunya, T.; Nakata, T. J. Org. Chem. 2002, 16, 5739; Li, X.; Ohtake, H.; Takahashi, H.; Ikegami, S. Syn. Lett. 2001, 1885; Hungerford, N. L.; Claridge, T. D.; Watterson, M. P.; Alpin, R. T.; Moreno, A.; Fleet, G. W. J. J. Chem. Soc. Perkin. Trans. 2000, 21, 3666; Harris, J. M.; Keraenen, M. D.; Nguyen, H.; Yound, V. G.; O'Doherty, G. A. Carbohydr. Res. 2000, 328, 17; Yuasa, H.; Tamura, J.; Hashimoto, H. J. Chem. Soc. Perkin. Trans. I 1990, 10, 2763. Sugar lactones 10 can be converted into enol ethers or gem-difluoro enol ethers of type 11 as follows. For non-fluoronated enol ethers, the readily available sugar lactones are reacted with the Tebbe reagent (Tebbe, F. N.; Parshall, G. W. Reddy, G. S. J. Am. Chem. Soc. 1977, 100, 3611) in mixtures of toluene and THF to afford the exo-methylene sugars (for example see RajanBabu, T. V.; Reddy, G. S. J. Org. Chem. 1986, 51, 5458). The carbohydrate gem-difluorenol ethers are readily prepared by reaction of the lactone precursors 10 with dibromodifluoromethane, tris(dimethylamino)phospine, and zinc, in a solvent such as THF (for example, see Houlton, J. S; Motherwell, W. B.; Ross, B. C.; Tozer, M. J.; Williams, D. J.; and Slawin, A. M. Z. Tetrahedron 1993, 49, 8087). The carbon-carbon bond-forming reaction at the anomeric position of the carbohydrate to generate C-glycosides of type 13 can be accomplished through Suzuki coupling of triflate 12 with an alkylboron reagent derived from the olefmated carbohydrate precursors 11 via hydroboration. For example, hydroboration of 11 with a suitable hydroborating agent such as 9-BBN or diborane, followed by in situ coupling with triflate 12, in the presence of a suitable palladium catalyst such as dichloro[1,1′-bis(diphenylphosphinoferrocene) palladium (II) dichloromethane adduct, in a solvent such as dimethylformamide, should proceed smoothly to provide C-glycosidics of type 13 (For example see Johns, B. A.; Pan, Y. T.; Elbein, A. D. Johnson, C. R. J. Am. Chem. Soc. 1997, 119, 4856). The hydroxyl-protected C-glycoside can then be globally deprotected to afford 14, or orthogonally deprotected to attach additional carbohydrate groups as described in Scheme B.

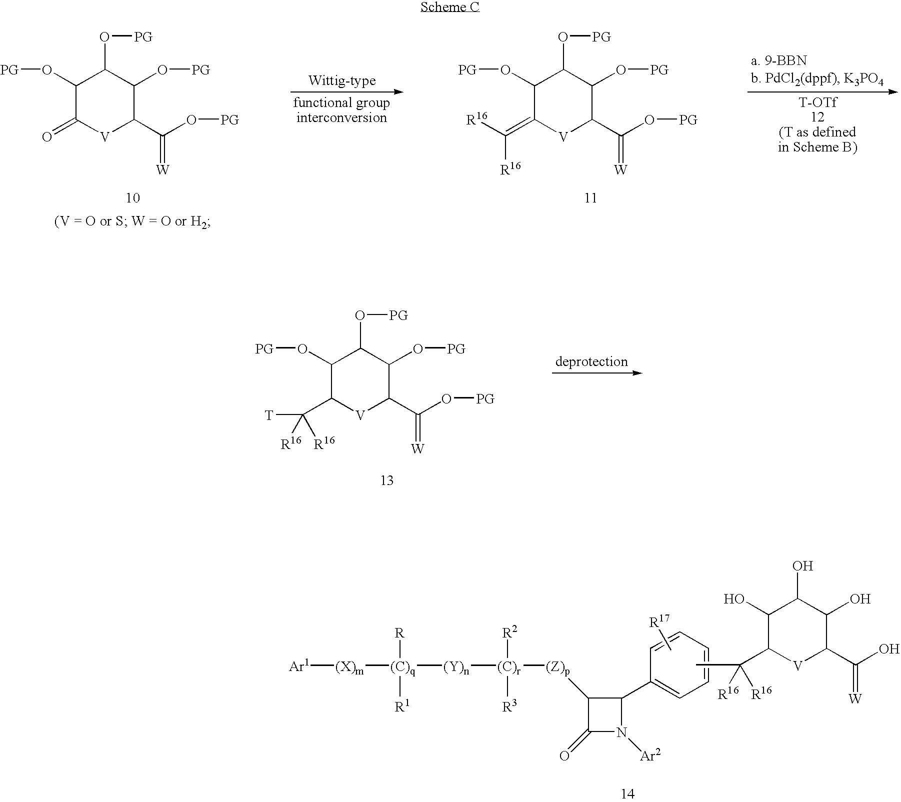

Reaction Schemes 1-19 illustrate the methods employed in the synthesis of the compounds of the present invention of structural Formula I. All substituents are as defined above unless indicated otherwise.

### EXAMPLE 1

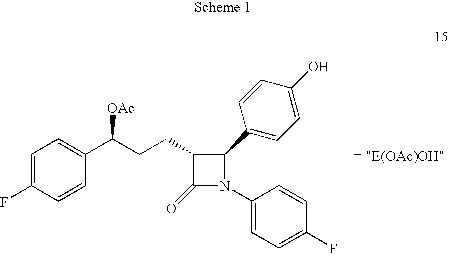

### Preparation of (1S)-1-(4-fluorophenyl)-3-[(2S,3R)-1-(4-fluorophenyl)-2-(4-hydroxyphenyl)-4-oxoazetidin-3-yl]propyl acetate (15) (also referred to herein as E(OAc)OH)

Intermediate 15 has been described previously, and can be prepared according to the methods outlined in Vaccaro. W. D.; Davis, H. R. Jr. Bioorg. Med. Chem. Lett. 1998, 8, 313.

### EXAMPLE 2

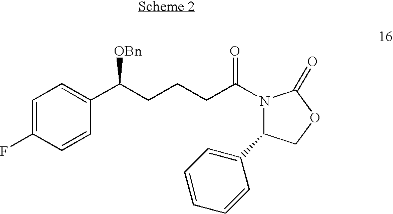

### Preparation of (4S)-3-[(5S)-5-(benzyloxy)-5-(4-fluorophenyl) pentanoyl]-4-phenyl-1,3-oxazolidin-2-one (16)

Sodium hydride (1.5 equiv. of a 60% dispersion in mineral oil) is added to a solution of (4S)-3-[(5S)-5-(4-fluorophenyl)-5-hydroxypentanoyl]-4-phenyl-1,3-oxazolidin-2-one (prepared according to WO 02/079174 A2, 2002) (1.0 equiv.) in the appropriate volume of dimethylformamide at 0° C. and the resulting mixture allowed to stir at r.t. for 40 min. The reaction is then cooled to 0° C. and benzylbromide (1.2 equiv.) is added and the reaction allowed to warm to r.t. with stirring until deemed complete. The reaction mixture is poured into water and extracted three times with EtOAc. The combined organic extract is washed with saturated aqueous sodium bicarbonate, water, dried (Na₂SO₄), filtered, and the filtrate concentrated in vacuo. Purification of the crude residue can be accomplished by employing a variety of chromatographic techniques to afford 16.

### EXAMPLE 3

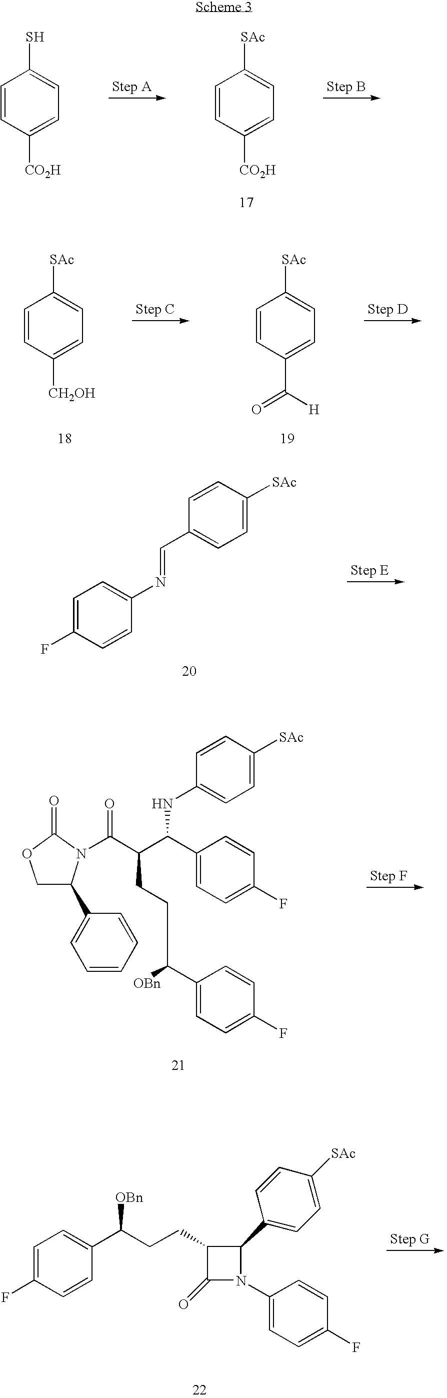 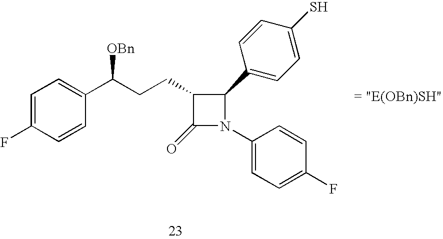 Step A:

### Preparation of 4-(acetylthio)benzoic acid (17)

The appropriate volume of acetic anhydride and pyridine (1:1) are added to 4-mercaptobenzoic acid (1.0 equiv.) at 0° C. with stirring, and the solution allowed to warm to r.t. and age until the reaction is deemed complete. The mixture is poured into water and extracted three times with EtOAc. The combined organic extracts are washed with brine, dried (MgSO₄), filtered, and the filtrate concentrated in vacuo. Purification of the crude residue can be accomplished by employing a variety of chromatographic techniques to afford 17.

Step B:

### Preparation of S-[4-(hydroxymethyl)phenyl] ethanethioate (18)

Borane-THF complex (2.5 equiv.) is added slowly to a solution of 17 (1.0 equiv.) in the appropriate volume of THF at −10° C. The reaction is allowed to warm to r.t. and stir until the reaction is deemed complete. The reaction mixture is quenched by the slow addition of the appropriate volume of water, diluted with 1 N aqueous hydrochloric acid and extracted three times with EtOAc. The combined organic extracts are washed with brine, dried (Na₂SO₄), filtered, and the filtrate concentrated in vacuo. The crude residue may be purified by a variety of chromatographic techniques to afford 18.

Step C:

### Preparation of S-(4-formylphenyl) ethanethioate (19)

Compound 19 can be prepared according to the following procedure (for example, see Shiozaki, M. J. Org. Chem. 1991, 56, 528). A solution of dimethyl sulfoxide (2.3 equiv.) is added to a solution of oxalyl chloride (1.6 equiv.) in the appropriate volume of DCM at −78° C. and the resulting solution allowed to stir 15 min. A solution of alcohol 18 (1.0 equiv.) in DCM is added dropwise to the above reaction mixture via syringe. After 45 min, triethylamine (5.0 equiv.) is added and the solution allowed to stir at −78° C. for 15 min, after which time the cooling bath is removed and the reaction aged at r.t. until deemed complete. The reaction mixture is quenched with water, poured into saturated aqueous sodium bicarbonate, and extracted three times with EtOAc. The combined organic extract is washed with brine, dried (Na₂SO₄), filtered, and the filtrate concentrated in vacuo. The crude residue can then be purified by employing a variety of chromatographic techniques to afford 19.

Step D:

### Preparation of S-(4-{(E)-[(4-fluorophenyl)imino]methyl}phenyl) ethanethioate (20)

A mixture of 19 (1.0 eqiuv.) and 4-fluoroaniline (1.0 equiv.) are heated at reflux in the appropriate volume of benzene with azeotrophic removal of water. When the reaction is deemed complete, the reaction mixture is cooled to r.t. and the volatiles evaporated. The crude residue may be purified by a variety of chromatographic techniques to afford 20.

Step E:

### Preparation of S-{4-[(1S,2R,5S)-5-(benzyloxy)-1,5-bis(4-fluorophenyl)-2-{[(4S)-2-oxo-4-phenyl-1,3-oxazolidin-3-yl]carbonyl}pentyl)amino]phenyl}ethanethioate (21)

Titanium tetrachloride (1.05 equiv.) is added dropwise to a solution of 16 (1.0 equiv.) in the appropriate volume of toluene at −70° C. After 45 min, DIPEA (2.0 equiv.) is added dropwise and the resulting mixture stirred at −0° C. for 2 h. A solution of 20 in the appropriate volume of DCM is added dropwise to the above reaction mixture while maintaining the internal temperature below −50° C. The resulting mixture is allowed to stir at −60° C. until the reaction is deemed complete. The reaction is quenched by the slow addition of the appropriate volume of acetic acid. After 30 min, the reaction is poured into 2 N H₂SO₄ at 0° C., and after 30 min, EtOAc is added and the biphasic mixture is stirred vigorously for 30 min. The organic layer is separated, and the aqueous phase re-extracted two times with EtOAc. The combined organic extracts are washed with saturated aqueous sodium bicarbornate, brine, dried (Na₂SO₄), filtered, and the filtrate concentrated in vacuo. The crude residue may be purified by a variety of chromatographic techniques to afford 21.

Step F:

### Preparation of S-{4-[(2S,3R)-3-[(3S)-3-(benzyloxy)-3-(4-fluorophenyl)propyl]-1-fluorophenyl)-4-oxoazetidin-2-yl]phenyl} ethanethioate (22)

N,O-bis(trimethylsilyl)acetamide (1.7 equiv.) is added to a solution of 21 (1.0 equiv.) in the appropriate volume of toluene and the resulting solution heated to 90° C. for approximately 2 h, then cooled to 65° C. Tetrabutyl ammonium fluoride hydrate (0.05 equiv.) is added and the reaction aged until deemed complete. The reaction is quenched with the appropriate volume of methanol, and the volatiles evaporated. The crude residue may be purified by a variety of chromatographic techniques to afford 22.

Step G:

### Preparation of (3R,4S)-3-[(3S)-3-(benzyloxy)-3-(4-fluorophenyl)propyl]-1-(4-fluorophenyl)-4-(4-mercaptophenyl)azetidin-2-one (23) (also referred to here in as E(OBn)SH)

Lithium hydroxide (4.0 equiv.) is added to a solution of 22 (1.0 equiv.) in an appropriate volume of water/THF (0.5:1) and the resulting mixture allowed to stir at r.t. until the reaction is deemed complete. The reaction mixture is concentrated then poured into saturated aqueous ammonium chloride and extracted three times with EtOAc. The combined organic extracts are washed with brine, dried (Na₂SO₄), filtered, and the filtrate concentrated in vacuo to afford 23, which may be purified further using a variety of chromatographic techniques.

### EXAMPLE 4

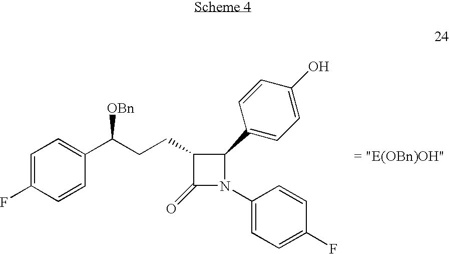

### Preparation of (3R,4S)-3-[(3S)-3-benzyloxy)-3-(4-fluorophenyl)propyl]-1-(4-fluorphenyl)-4-(4-hydroxyphenyl)azetidin-2-one (24) (also referred to herein as E(OBn)OH)

Compound 24 can be prepared from (4-fluorophenyl)amine and 4-formylphenyl acetate by appropriate modification of the procedure described in Steps D-G, Scheme 3.

### EXAMPLE 5

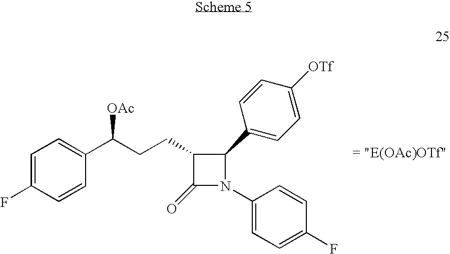

### Preparation of (1S)-1-(4-fluorophenyl)-3-[(3R,4S)-1-(4-fluorophenyl)-2-oxo-4-(4-{[(trifluoromethyl)sulfonyl]oxy}phenyl)azetidin-3-yl]propyl acetate (25) (also referred to herein as E(OAc)OTf)

DMAP (0.1 equiv.) and triethylamine (1.1 equiv.) are added to a solution of intermediate 15 (1.0 equiv.) in the appropriate volume of DCM. The reaction is cooled to −78° C., and trifluoromethane sulfonic anhydride is added dropwise via syringe. The reaction is allowed to stir at −78° C. until deemed complete. The mixture is poured into cold, saturated aqueous ammonium chloride, and extracted three times with EtOAc. The combined organic extracts are washed with brine, dried (MgSO₄), filtered, and the filtrate concentrated in vacuo. Purification of the crude residue can be accomplished by employing a variety of chromatographic techniques to afford 25.

### EXAMPLE 6

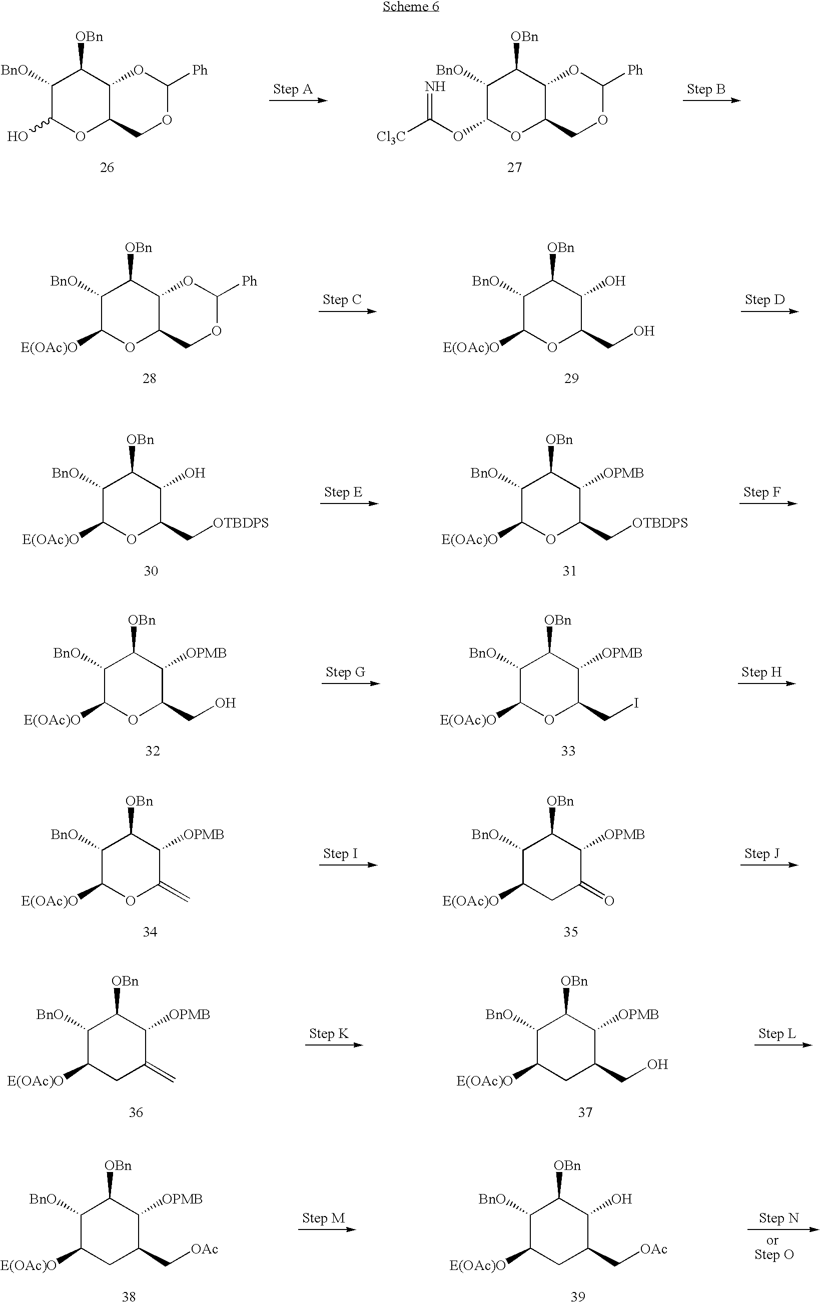

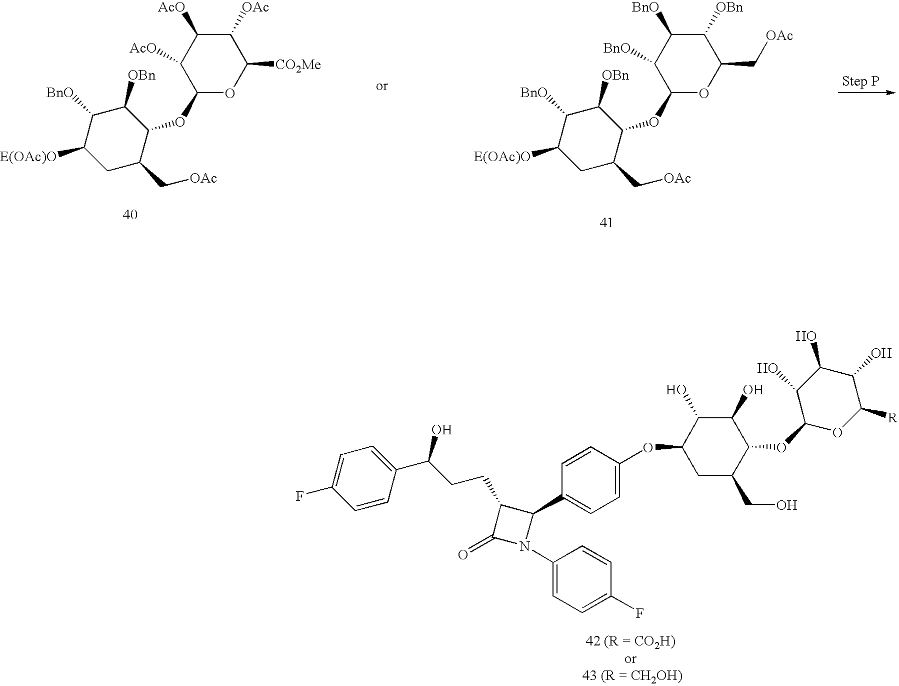

### Preparation of Compounds 42 and 43

Step A:

### Preparation of 2,3-di-O-benzyl-4,6-benzylidene-1-O-(2,2,2-trichloroethanimidoyl)-α-D-glucopyranose (27)

Trichloroacetimidate 27 can be prepared according to the following procedure (for example, see Xu, W.; Springfield, S. A.; Koh, J. T. Carb. Res. 2000, 169). 1,8-Diazabicyclo[5.4.0]undec-7-ene and trichloroacetonitrile are added to a solution of 26 (prepared according to Liotta, L. J.; Capotosts, R. D.; Garbitt, R. A.; Horan, B. M.; Kelly, P. J.; Koleros, A. P.; Brouillette, L. M.; Kuhn, A. M.; Targontsidis, S. Carb. Res. 2001, 331, 247) in the appropriate volume of DCM and the resulting solution allowed to stir at r.t. until the reaction is deemed complete. Purification of the crude residue can be accomplished by employing a variety of chromatographic techniques to afford 27.

Step B:

### Preparation of (1S)-3-[(2S,3R)-2-{4-[(2,3-di-O-benzyl-4,6-O-benzylidene-β-D-glucopyranosyl)oxy]phenyl}-1-(4-fluorophenyl)-4-oxoazetidin-3-yl]-1-(4-fluorophenyl)propyl acetate (28)

Compound 28 can be prepared according to the following procedure (for example, see Vaccaro. W. D.; Davis, H. R. Jr. Bioorg. Med. Chem. Lett. 1998, 8, 313). Boron trifluoride etherate (0.1 equiv.) is added to a −25° C. solution of 27 (1.0 equiv.) and 15 (1.2 equiv.) in the appropriate volume of DCM and the resulting reaction mixture maintained from −20° C. to 10° C. until the reaction is deemed complete. The mixture is poured into saturated aqueous ammonium chloride, and extracted three times with EtOAc. The combined organic extracts are washed with brine, dried (MgSO₄), filtered, and the filtrate concentrated in vacuo. Purification of the crude residue can be accomplished by employing a variety of chromatographic techniques to afford 28.

Step C:

### Preparation of (1S)-3-[(2S,3R)-2-{4-[(2,3-di-O-benzyl-β-D-glucopyranosyl)oxy]phenyl}1-(4-fluorophenyl)-4-oxoazetidin-3-yl]-1-(4-fluorophenyl)propyl acetate (29)

Compound 28 is treated with the appropriate volume of 0.01 N sulfuic acid until the reaction is deemed complete. The reaction mixture is poured into saturated aqueous sodium bicarbonate, and extracted three times with EtOAc. The combined organic extracts are washed with brine, dried (MgSO₄), filtered, and the filtrate concentrated in vacuo. Purification of the crude residue can be accomplished by employing a variety of chromatographic techniques to afford 29.

Step D:

### Preparation of (1S)-3-[(2S,3R)-2-[4-({2,3-di-O-benzyl-6-O-[tert-butyl(diphenyl)silyl]-β-D-glucopyranosyl}oxy)phenyl]-1-(4-fluorophenyl)-4-oxoazetidin-3-yl]-1-(4-fluorophenyl)propyl acetate (30)

Compound 30 can be prepared according to the following procedure (for example, see Tokutake, S.; Uchida, R.; Kotani, K.; Saito, K.; Yamaji, N. Carb. Res. 1993, 238, 109). Tert-Butylchlordiphenylsilane (4.0 equiv.) is add to a solution of 29 (1.0 equiv.) and imidazole (12 equiv.) in the appropriate volume of dimethylformamide and the resulting solution allowed to stir at r.t. until the reaction is deemed complete. Toluene is added and the mixture is washed with water, brine, dried (Na₂SO₄), filtered, and the filtrate concentrated in vacuo. The crude residue can then be purified by employing a variety of chromatographic techniques to afford 30.

Step E:

### Preparation of (1S)-3-[(2S,3R)-2-(4-{[2,3-di-O-benzyl-6-O-[tert-butyl(diphenyl)silyl]-4-O-(4-methoxybenzyl)-β-D-glucopyranosyl]oxy}phenyl)-1-(4-fluorophenyl)-4-oxoazetidin-3-yl]-1-(4-fluorophenyl)propyl acetate (31)

Compound 31 can be prepared according to the following procedure (for example, see Reddy, K. K.; Saady, M.; Falck, J. R. J. Org. Chem. 1995, 60, 3385). 4-methoxybenzyltrichloroacetimidate (2.5 equiv.) and triphenylcarbenium tetrafluoroborate (0.03 equiv.) are added to a solution of 30 in the appropriate volume of anhydrous ether and the resulting solution allowed to stir at r.t. until the reaction is deemed complete. The reaction mixture is then poured into 10% aqueous sodium bicarbonate, and extracted three times with EtOAc. The combined organic extracts are washed with brine, dried (MgSO₄), filtered, and the filtrate concentrated in vacuo. Purification of the crude residue can be accomplished by employing a variety of chromatographic techniques to afford 31.

Step F:

### Preparation of (1S)-3-[(2S,3R)-2-(4-{[2,3-di-O-benzyl-4-O-(4-methoxybenzyl)-β-D-glucopyranosyl]oxy}phenyl)-1-(4-fluorophenyl)-4-oxoazetidin-3-yl]-1-(4-fluorophenyl)propyl acetate (32)

TBAF (2.0 equiv.) is added to a solution of 31 in THF (1.0 equiv.) and the resulting solution allowed to stir at r.t. until the reaction is deemed complete. Purification of the crude residue can be accomplished by employing a variety of chromatographic techniques to afford 32.

Step G:

### Preparation of (1S)-3-[(2S,3R)-2-(4-{[2,3-di-O-benzyl-6-deoxy-6-iodo-4-O-(4-methoxybenzyl)-β-D-glucopyranosyl]oxy}phenyl)-1-(4-fluorophenyl)-4-oxoazetidin-3-yl]-1-(4-fluorophenyl)propyl acetate (33)

Iodide 33 can be prepared according to the following method (for example, see Sollogogoub, M.; Pearce, A. J.; Herault, A.; and Sinay, P. Tetrahedron: Asymmetry 2000, 11, 283). Imidazole (3.0 equiv.), triphenylphosphine (1.5 eqiv.) and iodine (1.1 equiv.) are added to a solution of 32 (1.0 equiv.) in the appropriate volume of anhydrous toluene at r.t. The reaction mixture is allowed to stir between r.t. and 70° C. until deemed complete. Upon cooling to r.t. the reaction is quenched with saturated aqueous sodium thiosulfate, and, after stirring for 5 min, is extracted with EtOAc. The organic extract is washed with water, dried (MgSO₄), filtered, and the filtrate concentrated in vacuo. The crude residue can then be purified by employing a variety of chromatographic techniques to afford 33.

Step H:

### Preparation of (1S)-3-[(2S,3R)-2-(4-{[2,3-di-O-benzyl-6-deoxy-4-O-(4-methoxybenzyl)-β-D-xylo-hex-5-enopyranosyl]oxy}phenyl)-1-(4-fluorophenyl)-4-oxoazetidin-3-yl]-1-(4-fluorophenyl)propyl acetate (34)

Alkene 34 can be prepared according to the following method (for example, see Sollogogoub, M.; Pearce, A. J.; Herault, A.; and Sinay, P. Tetrahedron: Asymmetry 2000, 11, 283). Sodium hydride (10 equiv., 60% dispersion in mineral oil) is added to a vigorously stirred solution of iodide 33 (1.0 equiv.) in the appropriate volume of anhydrous dimethylformamide at r.t. Upon completion, the reaction mixture is cooled to 0° C. and quenched via the slow addition of the appropriate volume of methanol. The solvent is removed in vacuo and the residue partitioned between DCM and water. The aqueous layer is extracted three times with DCM and the combined extract is washed with brine, dried (MgSO₄), filtered, and the filtrate concentrated in vacuo. The crude residue can then be purified by employing a variety of chromatographic techniques to afford 34.

Step I:

### Preparation of (1S)-3-[(2S,3R)-2-[4-({(1R,2S,3R,4S)-2,3-bis(benzyloxy)-4-[(4-methoxybenzyl)oxy]-5-oxocyclohexyl}oxy)phenyl]-1-(4-fluorophenyl)-4-oxoazetidin-3-yl]-1-(4-fluorophenyl)propyl acetate (35)

Ketone 35 can be prepared according to the following method (for example see Boyer, F-D; and Lallemand, J.-Y. Tetrahedron 1994, 50, 10433). Mercury (II) acetate (1.12 equiv.) and acetic acid (6.0 equiv.) are added to a solution of alkene 34 (1.0 equiv.) in the appropriate volume of water-acetone (1:2) and the resulting mixture allowed to stir at reflux until the reaction is deemed complete. After cooling to r.t., the organic solvents are evaporated under reduced pressure and the aqueous phase extracted three times with DCM. The combined organic extracts are washed with brine, dried (MgSO₄), filtered, and the filtrate concentrated in vacuo. Purification of the crude residue can be accomplished by employing a variety of chromatographic techniques to afford 35.

Step J:

### Preparation of (1S)-3-[(2S,3R)-2-[4-({(1R,2S,3S,4R)-2,3-bis(benzyloxy)-4-[(4-methoxybenzyl)oxy]-5-methylenecyclohexyl}oxy)phenyl]-1-(4-fluorophenyl)-4-oxoazetidin-3-yl]-1-(4-fluorophenyl)propyl acetate (36)

Alkene 36 can be prepared according to the following procedure (for example see Sollogogoub, M.; Pearce, A. J.; Herault, A.; and Sinay, P. Tetrahedron: Asymmetry, 2000, 11, 283) Pyridine (0.18 equiv.) and then the Tebbe reagent (3.0 equiv.) (Tebbe, F. N.; Parshall, G. W.; Reddy, G. S. J. Am. Chem. Soc. 1978, 100, 3611) are added to a solution of 35 (1.0 equiv.) in the appropriate volume of anhydrous toluene/THF at −45° C. under argon. The reaction is then allowed to stir at −45° C. for 1 h, 0° C. for 1 h, and finally at r.t. until the reaction is deemed complete. The reaction mixture is cooled to 0° C. and aqueous sodium hydroxide (15%) is added dropwise with caution. The mixture is warmed to r.t. and diluted with DCM. After stirring for 15 min, the mixture is filtered through Celite® and MgSO₄, washing with DCM. The filtrate is concentrated in vacuo and the crude residue purified by employing a variety of chromatographic techniques to afford 36.

Step K:

### Preparation of (1S)-3-[(2S,3R)-2-[4-({(1R,2S,3S,4R,5R)-2,3-bis(benzyloxy)-5-(hydroxymethyl)-4-[(4-methoxybenzyl)oxy]cyclohexyl}oxy)phenyl]-1-(4-fluorophenyl)-4-oxoazetidin-3-yl]-1-(4-fluorophenyl)propyl acetate (37)

Alcohol 37 can be prepared according to the following method (for example see Sollogogoub, M.; Pearce, A. J.; Herault, A.; and Sinay, P. Tetrahedron: Asynmmetry 2000, 11, 283). BH₃.THF (2.0 equiv.) is added to a solution of 36 (1.0 equiv.) in the appropriate volume of anhydrous THF at r.t. under argon. The reaction is allowed to stir at r.t. until deemed complete. The appropriate volume of ethanol, aqueous sodium hydroxide (3 M), and aqueous hydrogen peroxide (30%) are added and the mixture allowed to stir at r.t. until the oxidation is complete. The reaction mixture is poured into ice-water and stirred for approximately 5 min. The aqueous layer is extracted three times with DCM and the combined organic extracts dried (MgSO₄), filtered, and the filtrate concentrated in vacuo. The crude residue can then be purified by employing a variety of chromatographic techniques to afford 37.

Step L:

### Preparation of {(1R,2R,3S,4S,5R)-5-{4-[(2S,3R)-3-[(3S)-3-(acetyloxy)-3-(4-fluorophenyl)propyl]-1-(4-fluorophenyl)-4-oxoazetidin-2-yl]phenoxy}-3,4-bis(benzyloxy)-2-[(4-methoxybenzyl)oxy]cyclohexyl}methyl acetate (38)

Compound 38 can be prepared by appropriate modification of the procedure described in Step A, Scheme 3.

Step M:

### Preparation of [(1R,2R,3S,4S,5R)-5-{4-[(2S,3R)-3-[(3S)-3-(acetyloxy)-3-(4-fluorophenyl)propyl]-1-(4-fluorophenyl)-4-oxoazetidin-2-yl]phenoxy}-3,4-bis(benzyloxy)-2-hydroxycyclohexyl]methyl acetate (39)

Compound 39 can be prepared according to the following procedure (for example, see Reddy, K. K.; Saady, M.; Falck, J. R. J. Org. Chem. 1995, 60, 3385). 2,3-Dichloro-5,6-dicyano-1,4-benzoquinone (2.0 equiv.) is added to a solution of 38 in the appropriate volume of DCM-water (20:1) and the resulting solution allowed to stir at r.t. until the reaction is deemed complete. The reaction mixture is then poured into 10% aqueous sodium bicarbonate, and extracted three times with DCM. The combined organic extracts are washed with brine, dried (MgSO₄), filtered, and the filtrate concentrated in vacuo. Purification of the crude residue can be accomplished by employing a variety of chromatographic techniques to afford 39.

Step N:

### Preparation of (1R,2S,3S,6R)-4-{4-[(2S,3R)-3-[(3S)-3-(acetyloxy)-3-(4-fluorophenyl)propyl]-1-(4-fluorophenyl)-4-oxoazetidin-2-yl]phenoxy}-6-[(acetyloxy)methyl]-2,3-bis(benzyloxy)cyclohexyl methyl 2,3,4-tri-O-acetyl-β-D-glucopyranosiduronate (40)

Compound 40 can be prepared from 39 and methyl 2,3,4-tri-O-acetyl-1-O-2,2,2-trichloroethanimidoyl)-D-glucopyranuronate by appropriate modification of the procedure described in Step B above.

Step O:

### Preparation of 41

Compound 41 can be prepared from 39 and 6-O-acetyl-2,3,4-tri-O-benzyl-1-O-(2,2,2-trichloroethanimidoyl)-α-D-glucopyranose (prepared according to Wang, Y.; Mao, J.; Cai, M. Synth. Commun. 1999, 29, 2093) by appropriate modification of the procedure described in Step B above.

Step P:

### Preparation of (1R,2R,3R,4R,6R)-4-(4-{(2S,3R)-1-(4-fluorophenyl)-3-[(3S)-3-(4-fluorophenyl)-3-hydroxypropyl]-4-oxoazetidin-2-yl}phenoxy)-2,3-dihydroxy-6-(hydroxymethyl)cyclohexyl D-glucopyranosiduronic acid (42) or (1R,2R,3R,4R,6R)-4-(4-{(2S,3R)-1-(4-fluorophenyl)-3-[(3S)-3-(4-fluorophenyl)-3-hydroxypropyl]-4-oxoazetidin-2-yl}phenoxy)-2,3-dihydroxy-6-(hydroxymethyl)cyclohexyl β-D-glucopyranoside (43)

Part A: Compound 40 or 41 (1.0 equiv.) and 10% Pd on carbon (20% by weight), in the appropriate volume of ethanol, acetic acid, or mixtures thereof, is hydrogenated at atmospheric pressure until the reaction is deemed complete. The resulting mixture is filtered through a short column of Celite®, eluting copiously with ethanol. The filtrate is concentrated in vacuo. Part B: Lithium hydroxide (10 equiv.) is added to a solution of either of the above products (1.0 equiv.) in THF-methanol-water (2:1:1) and the resulting solution allowed to stir at r.t. until the reaction is deemed complete. The reaction mixture is neutralized with aqueous 1 N hydrochloric acid, concentrated in vacuo, poured into water, and extracted three times with EtOAc. The combined organic extract is washed with brine, dried (Na₂SO₄), filtered, and the filtrate concentrated in vacuo. The crude residue can then be purified by employing a variety of chromatographic techniques to afford either 42 or 43.

### EXAMPLE 7

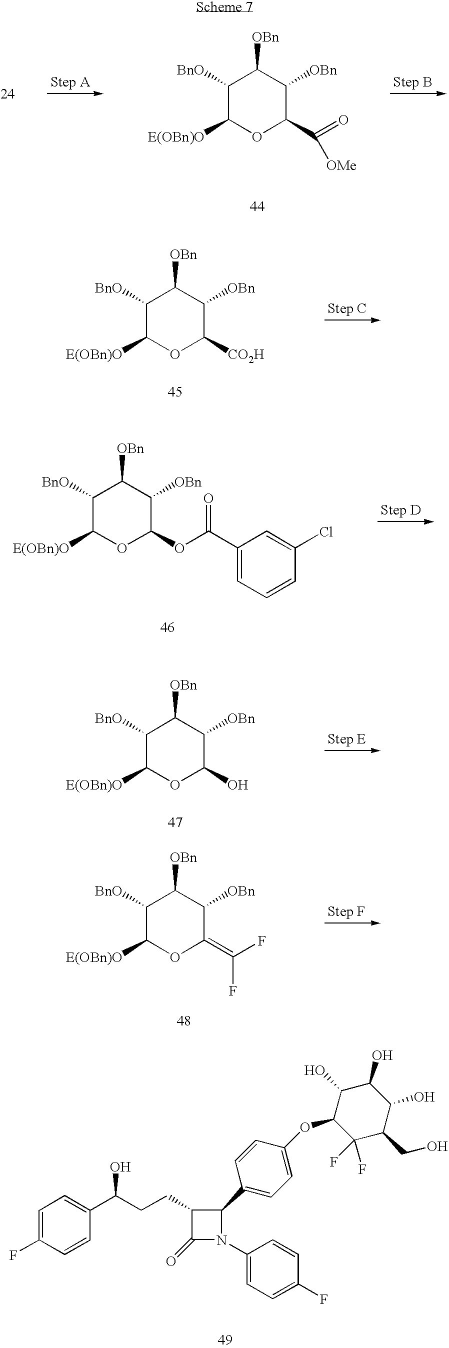

### Preparation of Compound 49

Step A:

### Preparation of 4-[(2S,3R)-3-[(3S)-3-(benzyloxy)-3-(4-fluorophenyl)propyl]-1-(4-fluorophenyl)-4-oxoazetidin-2-yl]phenyl methyl 2,3,4-tri-O-benzyl-β-D-glucopyranosiduronate (44)

Compound 44 can be prepared from 24 and methyl 2,3,4-tri-O-benzyl-1-O-2,2,2-trichloroethanimidoyl)-α-D-glucopyranuronate (prepared according to Schmidt, R. R.; Grundler, G. Synthesis, 1981, 885) by appropriate modification of the procedure described in Step B, Scheme 6.

Step B:

### Preparation of 4-[(2S,3R)-3-[(3S)-3-(benzyloxy)-3-(4-fluorophenyl)propyl]-1-(4-fluorophenyl)-4-oxoazetidin-2-yl]phenyl 2,3,4-tri-O-benzyl-β-D-glucopyranosiduronic acid (45)

Compound 45 can be prepared by appropriate modification of the procedure described in Step P (Part B), Scheme 6.

Step C:

### Preparation of 46

Compound 46 can be prepared according to the following procedure (for example, see Shiozaki, M. J. Org. Chem. 1991, 56, 528). 3-Chlorperoxybenzoic acid (1.2 equiv.) and 1,3-dicyclohexylcarbodiimide are added to a solution of acid 45 in the appropriate volume of DCM at 0 to 5° C. The resulting reaction mixture is allowed to warm to r.t. and age until the reaction is deemed complete. The reaction mixture is filtered and the filtrate concentrated in vacuo. The crude residue can then be purified by employing a variety of chromatographic techniques to afford 46.

Step D:

### Preparation of 47

Compound 47 can be prepared according to the following procedure (for example, see Shiozaki, M. J. Org. Chem. 1991,.56, 528). 0.1 M Sodium hydroxide (2.5 equiv.) is added to a solution of ester 46 (1.0 equiv.) in THF and the resulting solution allowed to stir at r.t. until the reaction is deemed complete. The reaction mixture is poured into water and extracted three times with EtOAc. The combined organic extract is washed with brine, dried (Na₂SO₄), filtered, and the filtrate concentrated in vacuo. The crude residue can then be purified by employing a variety of chromatographic techniques to afford 47.

Step E:

### Preparation of 4-[(2S,3R)-3-[(3S)-3-(benzyloxy)-3-(4-fluorophenyl)propyl]-1-(4-fluorophenyl)-4-oxoazetidin-2-yl]phenyl 2,3,4-tri-O-benzyl-6-deoxy-6,6-difluoro-β-D-xylo-hex-5-enopyranoside (48)

Part A: See Scheme 3, Step C. Part. B: Gem-difluoroenol ether 48 can be prepared from the product of Part A above, according to the following method (for example, see Houlton, J. S; Motherwell, W. B.; Ross, B. C.; Tozer, M. J.; Williams, D. J.; and Slawin, A. M. Z. Tetrahedron 1993, 49, 8087). Dibromodifluoromethane (4.5 equiv.) is added to a cooled solution (−20° C.) of the product from Part A above (1.0 equiv.) in THF at an appropriate concentration using a cooled syringe. Tris(dimethylamino)phoshine (4.5 equiv.) is then added to the vigorously stirred solution. The mixture is stirred at r.t. for 30 min, then zinc powder (4.5 equiv.) and another portion of tris(dimethylamino)phosphine (0.2 equiv.) are added and the mixture heated to reflux until the reaction is deemed complete. The mixture is allowed to cool to r.t. and ether is added. The ether layer is decanted and the residue washed with ether. The combined organic extracts are washed with a saturated copper sulfate solution until the solution remains blue, followed by water, and brine. The organics are dried (MgSO₄), filtered, and the filtrate concentrated in vacuo. Purification of the crude residue can be accomplished by employing a variety of chromatographic techniques to afford 48.

Step F:

### (3R,4S)-4-(4-{[(1S,3R,4R,5S,6R)-2,2-difluoro-4,5,6-trihydroxy-3-(hydroxymethyl)cyclohexyl]oxy}phenyl)-1-(4-fluorophenyl)-3-[(3S)-3-(4-fluorophenyl)-3-hydroxypropyl]azetidin-2-one (49)

Compound 49 can be prepared by appropriate modification of the general procedures described in Steps I-K, Scheme 6 and Step P (Part A) Scheme 6.

### EXAMPLE 8

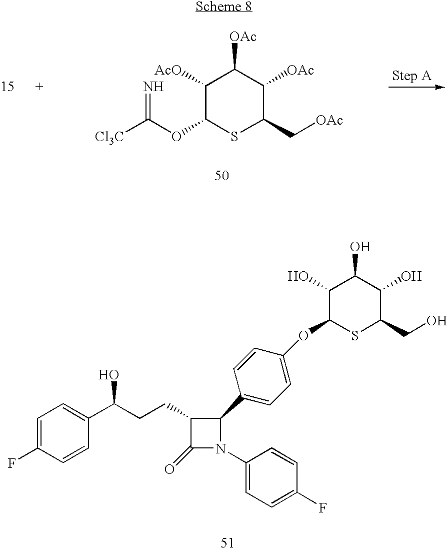

### Preparation of Compound 51

Step A:

### Preparation of 4-{(2S,3R)-1-(4-fluorophenyl)-3-[(3S)-3-(4-fluorophenyl)-3-hydroxypropyl]-4-oxoazetidin-2-yl}phenyl 5-thio-β-D-glucopyranoside (51)

Compound 51 can be prepared from 15 and 50 (prepared according to Izumi, M.; Suhara, Y.; Ichikawa, Y. J. Org. Chem. 1998, 63, 4811) by appropriate modification of the general procedures described in Step B, Scheme 6 and Step P (Part B) Scheme 6.

### EXAMPLE 9

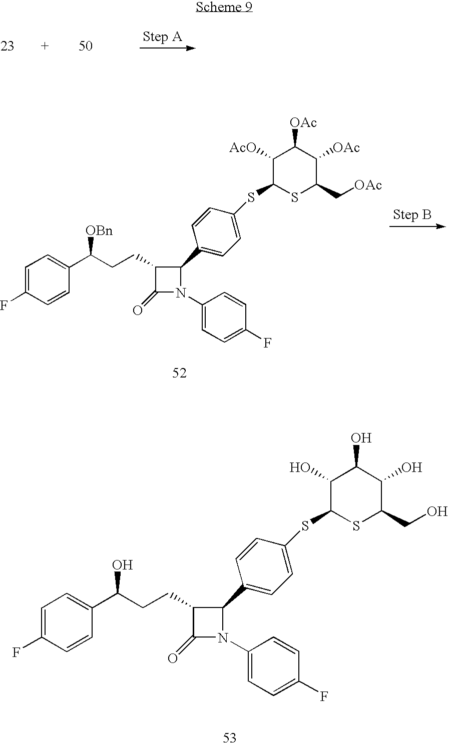

### Preparation of Compound 53

Step A:

### Preparation of 4-[(2S,3R)-3-[(3S)-3-(benzyloxy)-3-(4-fluorophenyl)propyl]-1-(4-fluorophenyl)-4-oxoazetidin-2-yl]phenyl 2,3,4,6-tetra-O-acetyl-1,5-dithio-β-D-glucopyranoside (52)

Compound 52 can be prepared from 23 and 50 by appropriate modification of the procedure described in Step B, Scheme 6.

Step B:

### Preparation of 4-{(2S,3R)-1-(4-fluorophenyl)-3-[(3S)-3-(4-fluorophenyl)-3-hydroxypropyl]-4-oxoazetidin-2-yl}phenyl 1,5-dithio-β-D-glucopyranoside (53)

Compound 53 can be prepared according to the following method (for example see Rodebaugh, R.; Debenham, J. S.; and Fraser-Reid, B. Tetrahedron Lett. 1996, 37, 5447). Ferric chloride (12 equiv.) is added to a solution of 52 in anhydrous DCM at 0° C. When deemed complete, the reaction is poured into water and extracted three times with DCM. The combined organic extracts are washed with brine, dried (MgSO₄), filtered, and the filtrate concentrated in vacuo. Purification of the crude residue can be accomplished by employing a variety of chromatographic techniques to afford the de-benzylated material. The product is then subjected to the reaction conditions described in Step P (Part B) Scheme 6 to afford compound 53.

### EXAMPLE 10

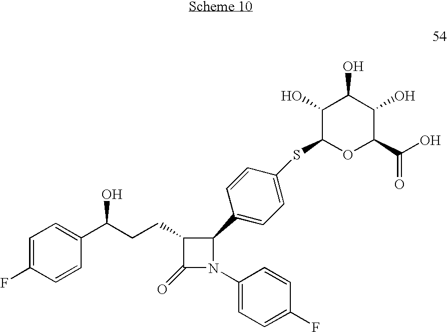

### Preparation of 4-{(2S,3R)-1-(4-fluorophenyl)-3-[(3S)-3-(4-fluorophenyl)-3-hydroxypropyl]-4-oxoazetidin-2-yl}phenyl 1-thio-β-D-glucopyranoside (54)

Compound 54 can be prepared from 23 and methyl 2,3,4-tri-O-acetyl-1-O-(2,2,2-trichloroethanimidoyl)-D-glucopyranuronate by appropriate modification of the procedures outlined in Steps A-B, Scheme 9.

### EXAMPLE 11

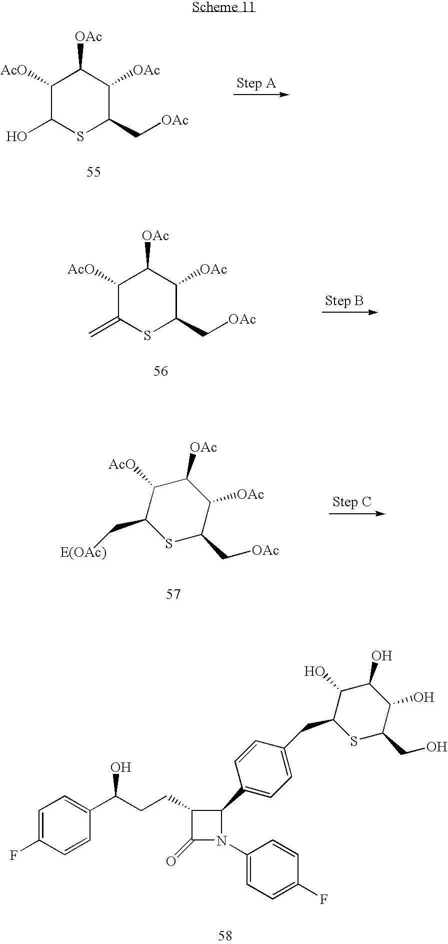

### Preparation of Compound 58

Step A:

### Preparation 56

Compound 56 can be prepared from 55 (prepared according to Izumi, M.; Suhara, Y.; Ichikawa, Y. J. Org. Chem. 1998, 63, 4811) according to the general procedure described in Step C, Scheme 3, followed by the general procedure described in Step J, Scheme 6.

Step B:

### Preparation of 57

Compound 57 can be prepared according to the following procedure (for example, see Johns, B. A.; Pan, Y. T.; Elbein, A. D. Johnson, C. R. J. Am. Chem. Soc. 1997, 119, 4856). 9-BBN (2.0 equiv.) is added to a solution of 56 (1.0 equiv.) in the appropriate volume of THF at r.t. and the resulting solution allowed to stir between room temperature and refluxing conditions for approximately 4 h. The reaction mixture is cooled to r.t. and 3 M aqueous K₃PO₄ (2.6 equiv.) is added. After approximately 15 min, a solution of triflate 25 (0.9 equiv.) and dichloro[1,1′-bis(diphenylphosphinoferrocene) palladium (II) DCM adduct (0.1 equiv.) in dimethylformamide is added via cannulation. The resulting mixture is allowed to stir at r.t. until the reaction is deemed complete. The mixture is poured into water, and extracted three times with EtOAc. The combined organic extracts are washed with brine, dried (MgSO₄), filtered, and the filtrate concentrated in vacuo. Purification of the crude residue can be accomplished by employing a variety of chromatographic techniques to afford 57.

Step C:

### Preparation of 58

Compound 58 can be prepared according to the general procedure described in Step P, Scheme 6.

### EXAMPLE 12

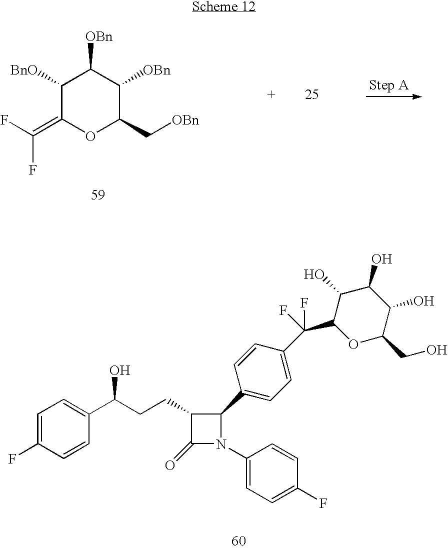 Step A:

### Preparation of 60

Compound 60 can be prepared from 59 (prepared according to Houlton, J. S; Motherwell, W. B.; Ross, B. C.; Tozer, M. J.; Williams, D. J.; and Slawin, A. M. Z. Tetrahedron 1993, 49, 8087) and 25 by appropriate modification of the general procedure described in Steps B-C, Scheme 11.

### EXAMPLE 13

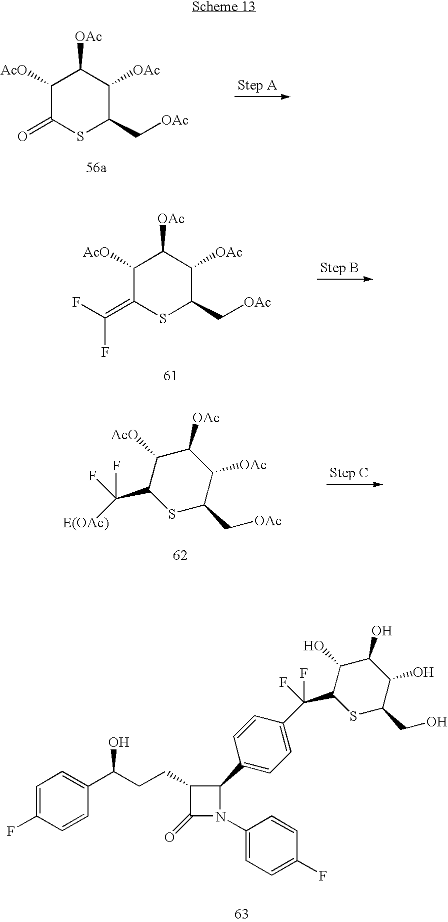

### Preparation of Compound 63

Step A:

### Preparation of 3,4,5,7-tetra-O-acetyl-2,6-anhydro-1-deoxy-1,1-difluoro-2-thio-D-gluco-hept-1-enitol (61)

Compound 61 can be prepared from 56a (see Scheme 11, Step A) according to the general procedure described in Step F (Part B), Scheme 7.

Step B:

### Preparation of 62

Compound 62 can be prepared by appropriate modification of the general procedure described in Step B, Scheme 11.

Step C:

### Preparation of 63

Compound 63 can be prepared by appropriate modification of the general procedure described in Steps O (Part B), Scheme 6.

### EXAMPLE 14

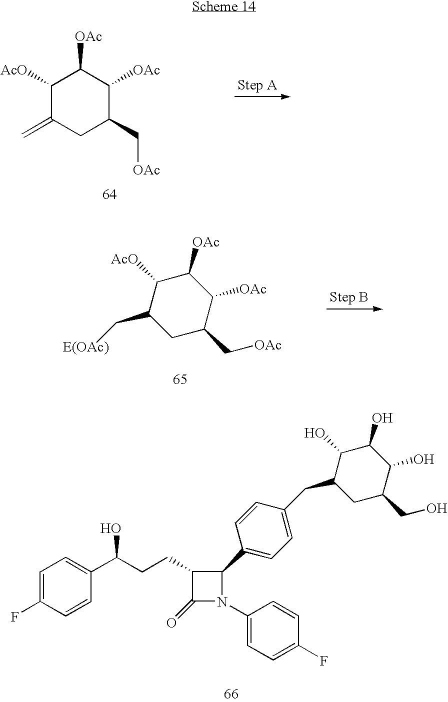

### Preparation of Compound 66

Step A:

### Preparation of (1R,2R,3S,4S,6R)-4-{4-[(2S,3R)-3-[(3S)-3-(acetyloxy)-3-(4-fluorophenyl)propyl]-1-(4-fluorophenyl)-4-oxoazetidin-2-yl]benzyl}-6-[(acetyloxy)methyl]cyclohexane-1,2,3-triyl triacetate (65)

Compound 65 can be prepared from 64 (prepared according to Gomez, A. M.; Danelon, G. O.; Valverde, S.; Lopez, J. C. J. Org. Chem. 1998, 63, 9626) by appropriate modification of the procedure outlined in Step B, Scheme 11.

Step B:

### Preparation of (3R,4S)-1-(4-fluorophenyl)-3-[(3S)-3-(4-fluorophenyl)-3-hydroxypropyl]-4-(4-{[(1S,2S,3R,4R,5R)-2,3,4-trihydroxy-5-(hydroxymethyl)cyclohexyl]methyl}phenyl)azetidin-2-one (66)

Compound 66 can be prepared from 65 by appropriate modification of the procedure outlined in Step P (Part B), Scheme 6.

### EXAMPLE 15

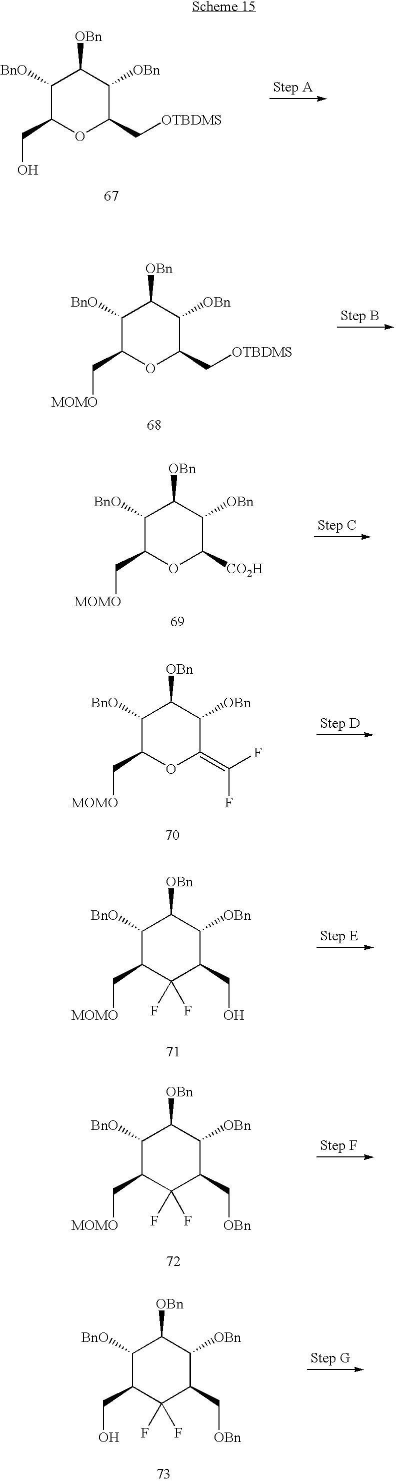

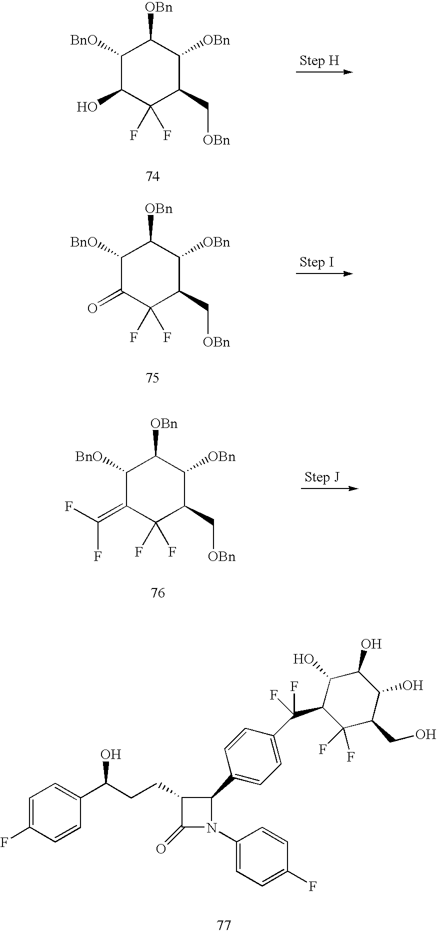

### Preparation of Compound 77

Step A:

### Preparation of 2,6-anhydro-3,4,5-tri-O-benzyl-1-deoxy-1,1-difluoro-7-O-(methoxymethyl)-L-gluco-hept-1-enitol (73)

Compound 68 can be prepared from 67 (prepared according to Nicolaou, K. C.; Florke, H.; Egan, M. G.; Barth, T.; Estevez, V. A. Tetrahedron Lett. 1995, 36, 1775) according to the following procedure (for example see Tokutake, S.; Uchida; R.; Kotani, K.; Saito, K.; Yamaji, N. Carb. Res. 1993, 238, 109). Chloromethyl methyl ether (5.0 equiv.) and DIPEA (5.0 equiv.) are added to a solution of 67(1.0 equiv.) in the appropriate volume of DCM and the mixture allowed to stir between r.t. and 60° C. until the reaction is deemed complete. The mixture is evaporated in vacuo and the final product 68 purified using a variety of chromatographic techniques.

Step B:

### Preparation of (69)

Compound 69 can be prepared from 68 by appropriate modification of the general procedure described in Step F, Scheme 6, followed by Step C, Scheme 3. The resulting aldehyde can then be subjected to the following reaction conditions to generate the corresponding acid (for example, see Shiozaki, M. J. Org. Chem. 1991, 56, 528). Sodiumperiodate (4.0 equiv.) and ruthenium oxide hydrate (0.13 equiv.) are added to a solution of the above aldehyde (1.0 equiv.) in the appropriate volume of acetonitrile-carbon tetrachloride-water (2:2:3) and the resulting mixture allowed to stir at room temperature until the reaction is deemed complete. The reaction mixture is poured into water and extracted three times with ethyl acetate. The combined organic extract is washed with brine, dried (Na₂SO₄), filtered, and the filtrate concentrated in vacuo. The crude residue can then be purified by employing a variety of chromatographic techniques to afford acid 69.

Step C:

### Preparation of 2,6-anhydro-3,4,5-tri-O-benzyl-1-deoxy-1,1-difluoro-7-O-(methoxymethyl)-L-gluco-hept-1-enitol (70)

Compound 70 can be prepared from 69 by appropriate modification of the general procedures described in Steps C-E, Scheme 7.

Step D:

### Preparation of {(1R,3S,4S,5S,6R)-4,5,6-tris(benzyloxy)-2,2-difluoro-3-[(methoxymethoxy)methyl]cyclohexyl}methanol (71)

Compound 71 can be prepared by appropriate modification of the general procedures described in Steps I-K, Scheme 6.

Step E:

### Preparation of [({(1R,2R,3R,5S,6S)-2,6-bis(benzyloxy)-3-[(benzyloxy)methyl]-4,4-difluoro-5-[(methoxymethoxy)methyl]cyclohexyl}oxy)methyl]benzene (72)

Compound 72 can be prepared by appropriate modification of the general procedure described in Scheme 2.

Step F:

### Preparation of {(1S,3R,4R,5R,6S)-4,5,6-tris(benzyloxy)-3-[(benzyloxy)methyl]-2,2-difluorocyclohexyl}methanol (73)

Compound 73 can be prepared according the following method (for example, see Hanessian, S.; Delorme, D.; Dufrense, Y. Tetrahedron Lea. 1984, 25, 2515). Trimethylsilyl bromide (4.0 equiv.) is added to a cooled (−30° C.) solution of 72 (1.0 equiv.) in the appropriate volume of DCM. The resulting solution is allowed to stir at −30° C. for one hour, then at 0° C. until the reaction is deemed complete. The mixture is poured into saturated aqueous sodium bicarbonate, and extracted three times with EtOAc. The combined organic extracts are washed with brine, dried (MgSO₄), filtered, and the filtrate concentrated in vacuo. Purification of the crude residue can be accomplished by employing a variety of chromatographic techniques to afford 73.

Step G:

### Preparation of (1S,3R,4R,5S,6S)-4,5,6-tris(benzyloxy)-3-[(benzyloxy)methyl]-2,2-difluorocyclohexanol (74)

Compound 74 can be prepared by appropriate modification of the general procedures described in Step C, Scheme 3, followed by oxidation to the acid as described in Step B above, followed by conversion to the tertiary alcohol as described in Steps C-D, Scheme 7.

Step H:

### Preparation of (3R,4R,5S,6R)-4,5,6-tris(benzyloxy)-3-[(benzyloxy)methyl]-2,2-difluorocyclohexanone (75)

Compound 75 can be prepared by appropriate modification of the general procedure described in Step C, Scheme 3.

Step I:

### Preparation of ({[(1R,2R,5S,6S)-5,6-bis(benzyloxy)-2-[(benzyloxy)methyl]-4-(difluoromethylene)-3,3-difluorocyclohexyl]oxy}methyl)benzene (76)

Compound 76 can be prepared according to the following method (for example, see Schwarz, S.; Thieme, I.; Kosemund, D.; Undeutsch, B.; Kummer, M.; Gorls, H.; Romer, W.; Kaufmann, G.; Elger, W.; Hillisch, A.; Schneider, B. Pharmazie 2001, 56, 843). tert-Butyl lithium (1.0 equiv.) is added to a −70° C. solution of diethyldifluoromethylphosphonate (1.0 equiv.) in the appropriate volume of ethylene glycol dimethyether/n-pentane (5:1) and the resulting solution allowed to stir for 15 min. A solution of ketone 75 (0.4 equiv.) in the appropriate volume of ethylene glycol dimethyether/n-pentane (5:1) is added. The reaction mixture is maintained at −70° C. for 30 min, then slowly distilled until the reaction mixture reaches 80° C., and finally heated to reflux until the reaction is deemed complete. After cooling to r.t., the reaction is quenched with water, filtered, and the filtrate concentrated in vacuo. Purification of the crude residue can be accomplished by employing a variety of chromatographic techniques to afford 76.

Step J:

### Preparation of (3R,4)-4-{4-[[(1S,3R,4R,5S,6S)-2,2-difluoro-5,6-trihydroxy-3-(hydroxymethyl)cyclohexyl](difluoro)methyl]phenyl}-1-(4-fluorophenyl)-3-[(3S)-3-(4-fluorophenyl)-3-hydroxypropyl]azetidin-2-one (77)

Compound 77 can be prepared by appropriate modification of the general procedures described in Steps B-C, Scheme 11.

### EXAMPLE 16

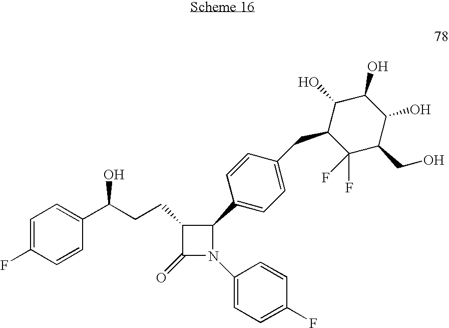 Preparation of Compound 78

### Preparation of (3R,4S)4-(4-{[(1S,3R,4R,5R,6S)-2,2-difluoro-4,5,6-trihydroxy-3-(hydroxymethyl)cyclohexyl]methyl}phenyl)-1-(4-fluorophenyl)-3-[(3S)-3-(4-fluorophenyl)-3-hydroxypropyl]azetidin-2-one (78)

Compound 78 can be prepared from 75 and 25 by appropriate modification of the general procedures described in Steps A-C, Scheme 11.

### EXAMPLE 17

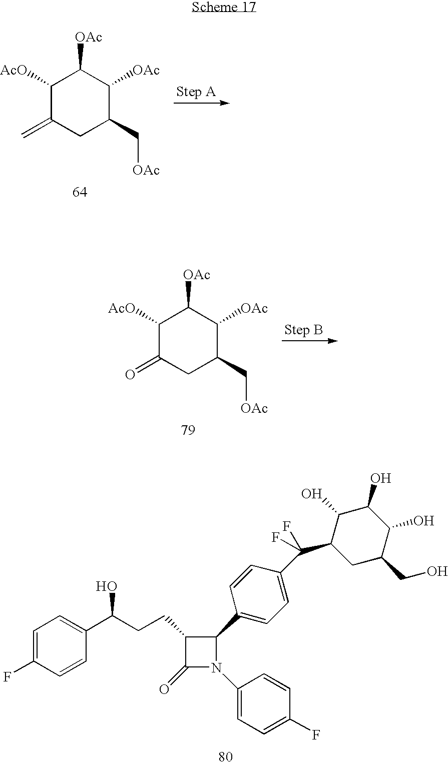 Preparation of Compound 80
Step A:

### Preparation of (1R,2S,3R,4R)-4-[(acetyloxy)methyl]-6-oxocyclohexane-1,2,3-triyl triacetate (79)

Ozone is bubbled through a solution of 64 (1.0 equiv.) in the appropriate volume of DCM at −78° C. until the reaction is deemed complete. Nitrogen is then bubbled through the solution until excess ozone is removed. Dimethyl sulfide (10 equiv.) is added and the solution allowed to warm to r.t. with stirring. The volatiles are evaporated and the product 79 may be purified using a variety of chromatographic techniques.

Step B:

### Preparation (3R,4S)-4-(4-{difluoro[(1R,2S,3S,4R,5R)-2,3,-trihydroxy-5-(hydroxymethyl)cyclohexyl]methyl}phenyl)-1-(4-fluorophenyl)-3-[(3S)-3-(4-fluorophenyl)-3-hydroxypropyl]azetidin-2-one (80)

Compound 80 can be prepared by appropriate modification of the general procedures described in Step I-J, Scheme 15.

### EXAMPLE 18

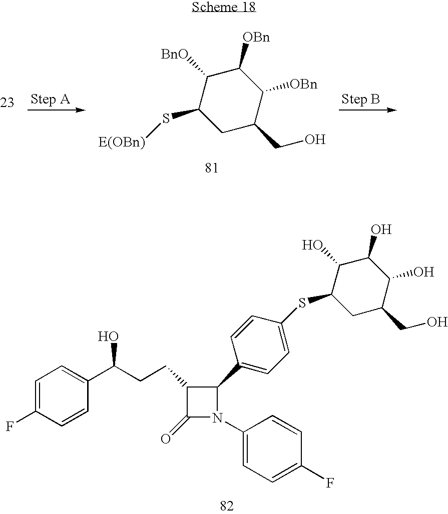 Preparation of Compound 82
Step A:

### Preparation (3R,4S)-3-[(3S)-3-(benzyloxy)-3-(4-fluorophenyl)propyl]-1-(4-fluorophenyl)-4-(4-{[(1R,2R,3S,4R,5R)-2,3,4-tris(benzyloxy)-5-(hydroxymethyl)cyclohexyl]thio}phenyl)azetidin-2-one (81)

Compound 81 can be prepared from 6-O-acetyl-2,3,4-tri-O-benzyl-1-O-(2,2,2-trichloroethanimidoyl)-α-D-glucopyranose (prepared according to Wang, Y.; Mao, J.; Cai, M. Synth. Commun. 1999, 29, 2093) and 23 by appropriate modification of the general procedures described in Steps B, Step P (part B), and Steps G-K, Scheme 6.

Step B:

### Preparation (3R,4S)-1-(4-fluorophenyl)-3-[(3S)-3-(4-fluorophenyl)-3-hydroxypropyl]4-(4-{[(1R,2R,3S,4R,5R)-2,3,4-trihydroxy-5-(hydroxymethyl)cyclohexyl]thio}phenyl)azetidin-2-one (82)

Compound 82 can be prepared by appropriate modification of the general procedures described in Step B (part A), Scheme 9.

What is claimed is:

1. compounds of Formula I 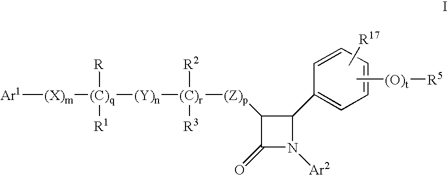
and the pharmaceutically acceptable salts and esters thereof, wherein
Ar¹ and Ar² are independently selected from the group consisting of aryl and R⁴-substituted aryl;
X, Y and Z are independently selected from the group consisting of —CH₂—, —CH(C₁₋₆alkyl)- and —C(C₁₋₆alkyl)₂-;
R is selected from the group consisting of —OR⁶, —O(CO)R⁶, —O(CO)OR⁹, —O(CO)NR⁶R⁷, a sugar residue, a disugar residue, a trisugar residue and a tetrasugar residue;
R1 is selected from the group consisting of hydrogen, C₁₋₆alkyl and aryl or R and R¹ together are oxo;
R² is selected from the group consisting of —OR⁶, —O(CO)R⁶, —O(CO)OR⁹ and —O(CO)NR⁶R⁷;
R³ is selected from the group consisting of hydrogen, —C₁₋₆alkyl and aryl or R² and R³ together are oxo; q, r and t are each independently selected from 0 and 1; m, n and p are each independently selected from 0, 1, 2, 3 and 4; provided that at least one of q and r is 1, and the sum of m, n, p, q are r is 1, 2, 3, 4, 5 or 6; and provided that when p is 0 and r is 1, the sum of m, q and n is 1, 2, 3, 4, or 5;
R⁴ is 1-5 substituents independently selected at each occurrence from the group consisting of: —OR⁶, —O(CO)R⁶, —O(CO)OR⁹, —O—C₁₋₅alkyl-OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O—C₁₋₁₀alkyl-COOR⁶, —O—C₁₋₁₀alkyl—CONR⁶R⁷ and fluoro;
R⁶, R⁷ and R⁸ are independently selected at each occurrence from the group consisting of hydrogen, C₁₋₆alkyl, aryl and aryl-substituted C₁₋₆alkyl;
R⁹ is independently selected from the group consisting of C₁₋₆alkyl, aryl and aryl-substituted C₁₋₆alkyl;
R⁵ is selected from
(a) —R¹⁰—R¹¹, wherein R¹⁰ is selected from the group consisting of —S—, —S(O)—, —SO₂— and —C₁₋₆ n-alkyl- substituted with one to three substituents selected from the group consisting of —C₁₋₆alkyl, —O(C₁₋₆alkyl), —CF₃, —OCF₃, —NR⁶R⁷ and —F;
(b) —R¹²—R¹³, wherein R¹² is selected from (i) a bond and (ii) a member selected from the group consisting of —S—, —S(O)—, —SO₂—, —C₁₋₆ n-alkyl-, and —C₁₋₆ n-alkyl-N(R⁶)—, wherein the alkyl group is unsubstituted or substituted with one to three substituents selected from the group consisting of —OH, oxo, —C₁₋₆alkyl, —O(C₁₋₆alkyl), —CF₃, —OCF₃, —NR⁶R⁷ and —F, and provided that when R¹² is a bond then t is 1;
R¹¹ is selected from the group consisting of a sugar residue, disugar residue, trisugar residue and tetrasugar residue;
R¹³ is selected from the group consisting of:
(a) a thiosugar residue selected from the group consisting of: 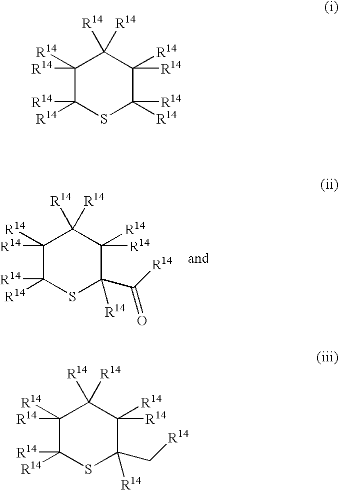
wherein R¹⁴ is independently selected at each occurrence from (i) a lining bond and (ii) a member of the group consisting of —F, —H, —C₁₋₆alkyl, —OC₁₋₆alkyl, —OCF₃, —OH, —O—PG, —OR¹¹ and —OR¹³, and provided that: (A) one and only one occurrence of R¹⁴ is a linking bond, (B ) an R¹⁴ adjacent to a carbonyl is not —F, and (C) no more than one occurrence of R¹⁴ is selected from —OR¹¹ and —OR¹³;
(b) a fluorosugar residue selected from the group consisting of: 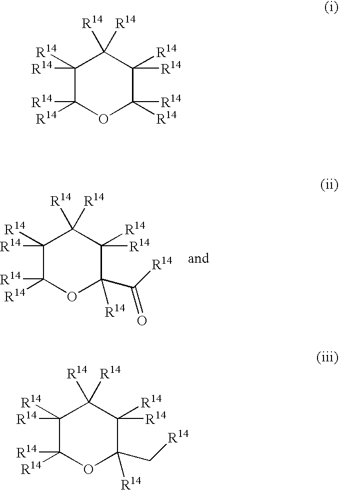
wherein R¹⁴ is independently selected at each occurrence from (i) a linking bond and (ii) a member of the group consisting of —F, —H, —CH₁₋₆alkyl, —OC₁₋₆alkyl, —OCF₃, —H, —O—PG, —OR¹¹ and —OR¹³, and provided that: (A) one and only one occurrence of R¹⁴ is a linking bond, (B) at least one occurrence of R¹⁴ is —F, (C) an R¹⁴ adjacent to a carbonyl is not —F, and (D) no more than one occurrence of R¹⁴ is selected from —OR¹¹ and —OR¹³; 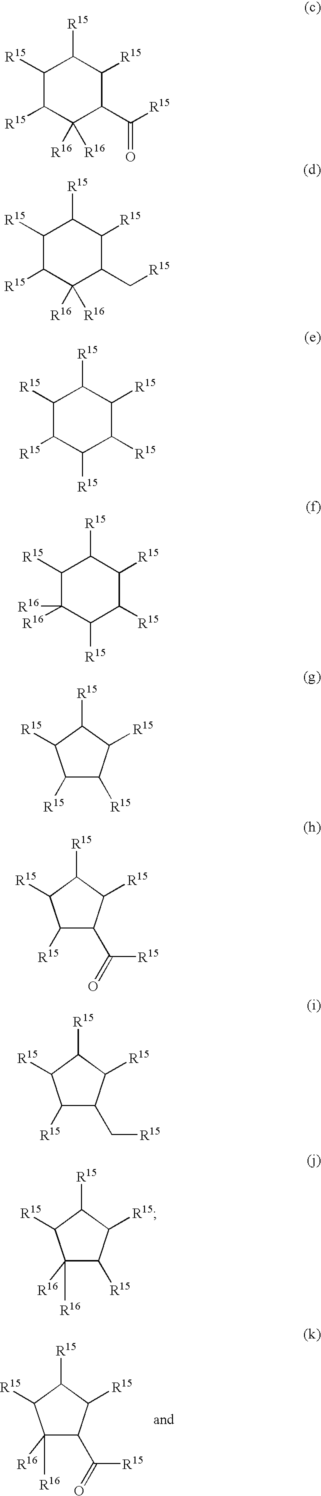 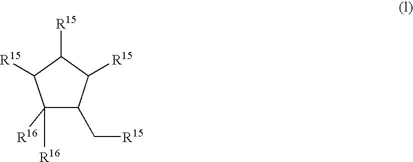
wherein R¹⁵ is independently selected at each occurrence from (i) a linking bond and (ii) a member of the group consisting of —H, —C₁₋₆alkyl, —OC₁₋₆alkyl, —OCF₃, —OH, —O—PG, —OR¹¹, —OR¹³, —SR¹¹, —SR¹³, —NR⁶R¹¹ and —NR⁶R¹³, and provided that: (A) one and only one occurrence of R¹⁵ is a linking bond and (B) no more than one occurrence of R¹⁵ is selected from —OR¹¹, —OR¹³ , —SR¹¹, —SR¹³, —NR⁶R¹¹ and —NR⁶R¹³;
R¹⁶ is independently selected at each occurrence from the group consisting of —H and —F;
PG is a hydroxyl protecting group;
and provided that R⁵ is comprised of no more than four of any combination of sugar residues and members within the definition of R¹³ linked together, and
R¹⁷ is selected from the group consisting of —H, —OH, —C₁₋₆alkyl, —OC₁₋₆alkyl, —CF₃, —CN, —NR⁶R⁷ and halogen.

2. The compound of claim 1 wherein the —(O)ₜ- R⁵ moiety is attached to the phenyl ring para to the azetidinone, and the R⁵ group is comprised of either —R¹⁰ or —R¹² and one or two of a combination of sugar residues and members within the definition of R¹³ linked together.

3. The compound of claim 1 of Formula Ia: 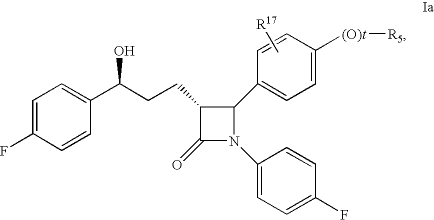
and the pharmaceutically acceptable salts and esters thereof.

4. The compound of claim 3 wherein the R⁵ group is comprised of one or two of a combination of sugar residues and members within the definition of R¹³ linked together.

5. The compound of claim 2 wherein t is one, R⁵ is —R¹²—R¹³, and R¹² is a bond.

6. The compound of claim 5 wherein R¹³ is a thiosugar.

7. The compound of claim 5 wherein R¹³ is 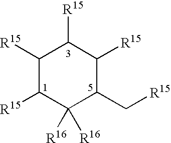
R¹⁵ at position 1 is a linking bond.

8. The compound of claim 7 selected from that wherein (a) all the remaining R¹⁵ groups are —H; and (b) R¹⁵ at position 4 is —OR¹¹ and the remaining R¹⁵ groups are —OH.

9. The compound of claim 2 wherein t is zero and R⁵ is —R¹⁰—R¹¹

10. The compound of claim 9 wherein R¹¹ is a sugar residue or a disugar residue.

11. The compound of claim 10 wherein R¹⁰ is selected from —S— and —CF₂—.

12. A method of reducing plasma cholesterol levels comprising administering a therapeutically effective amount of a compound of claim 1 to a patient in need of such treatment.

13. A method of treating hypercholesterolemia comprising administering a therapeutically effective amount of a compound of claim 1 to a patient in need of such treatment.

14. A method of treating atherosclerosis comprising administering a therapeutically effective amount of a compound of claim 1 to a patient in need of such treatment.

15. A method of reducing the risk for atherosclerosis comprising administering a prophylactically effective amount of a compound of claim 1 to a patient in need of such treatment.

16. A method of reducing the risk for having an atherosclerotic disease event comprising administering a prophylactically effective amount of a compound of claim 1 to a patient in at risk for such an event.

17. A pharmaceutical composition comprising a compound of claim 1 and a pharmaceutically acceptable carrier.
